SEQUENCE LISTING (27 sequences)

SEQ ID NO: 1 - protein, 20 aa, homo sapiens
  PEPTIDE (1)..(20): aa 153-172 of NKp46 (SEQ ID NO5 herein)
FLLLKEGRSSHVQRGYGKVQ

SEQ ID NO: 2 - protein, 28 aa, homo sapiens
  PEPTIDE (1)..(28): derived from NKp30 amino acids 56-83
RDEVVPGKEVRNGTPEFRGRLAPLASSR

SEQ ID NO: 3 - protein, 20 aa, homo sapiens
  PEPTIDE (1)..(20): corresponds to amino acids 56-75 of NKp30
RDEVVPGKEVRNGTPEFRGR

SEQ ID NO: 4 - protein, 24 aa, homo sapiens
  PEPTIDE (1)..(20): amino acids 61-80 of NKp44
KKGWCKEASALVCIRLVTSSKPRT

SEQ ID NO: 5 - protein, 304 aa, homo sapiens <s300> NCBI/CAA04714 1998-09-22 (1)..(304)
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 6 - protein, 287 aa, homo sapiens <s300> NCBI/CAA06872 1998-09-22 (1)..(287)
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDHALWDHTAQNLLRMGLAFLVLVALVWFLVEDWLSRKRTRERASRASTWEGRRRLNTQTL

SEQ ID NO: 7 - protein, 209 aa, homo sapiens <s300> NCBI/CAA06873 1998-09-22 (1)..(209)
MSSTLPALLCVGLCLSQRISAQQQMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 8 - protein, 192 aa, homo sapiens <s300> NCBI/CAA06874 1998-09-22 (1)..(192)
MSSTLPALLCVGLCLSQRISAQQQMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDHALWDHTAQNLLRMGLAFLVLVALVWFLVEDWLSRKRTRERASRASTWEGRRRLNTQTL

SEQ ID NO: 9 - protein, 488 aa, artificial
  conjugate of leader peptide, D1 and D2 domains of NKp46 with Fc domain
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 364 aa, artificial
  conjugate of CD5 leader peptide and D1 of NKp46 with Fc domain
MGMPMGSLQPLATLYLLGMLVASCLGRLRVPQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 393 aa, artificial
  conjugate of CD5 leader peptide and D2 domain of NKp46 with Fc domain
MGMPMGSLQPLATLYLLGMLVASCLGRLRVPYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDTWGTYLLTTETGLQKDHALWDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 201 aa, homo sapiens <s300> NCBI/AAH52582 2004-06-30 (1)..(201)
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 13 - protein, 382 aa, artificial
  conjugate of CD5 leader peptide and D1domain of NKp30 with Fc domain
MGMPMGSLQPLATLYLLGMLVASCLGRLRVPLWVSQPLEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 276 aa, homo sapiens <s300> NCBI/CAB39168 1999-03-15 (1)..(276)
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTPWTPRDLVSSQTQTQSCVPPTAGARQAPESPSTIPVPSQPQNSTLRPGPAAPIALVPVFCGLLVAKSLVLSALLVWWGDIWWKTVMELRSLDTQKATCHLQQVTDLPWTSVSSPVEREILYHTVARTKISDDDDEHTL

SEQ ID NO: 15 - protein, 434 aa, artificial [sequence omitted]

SEQ ID NO: 16 - protein, 326 aa, artificial
  conjugate of CD5 leader peptide and DS domain of NKP44 with Fc domain
MGMPMGSLQPLATLYLLGMLVASCLGRLRVPSPASASTQTSWTPRDLVSSQTQTQSCVPPTAGARQAPESPSTIPVPSQPQNSTLRPGPAAPDPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 376 aa, artificial
  conjugate of leader peptide,and DL domain of Nkp44 with Fc domain
MGMPMGSFQPLATLYLLGMLVASCLGRLRVPQSKAQVLQSVAGQTLTVRCQYPPTGSLYEKKGWCKEASALVCIRLVTSSKPRTVAWTSRFTIWDDPDAGFFTVTMTDLREEDSGHYWCRIYRPSDNSVSKSVRFYLVVSPADPEPKSSDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 914 bp, homo sapiens <s300> NCBI/AJ001383 1998-09-22 (1)..(914)
tgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcggatgggcctggcctttctagtcctggtggctctagtgtggttcctggttgaagactggctcagcaggaagaggactagagagcgagccagcagagcttccacttgggaaggcaggagaaggctgaacacacagactctttga

SEQ ID NO: 19 - DNA, 1506 bp, artificial
  DNA sequence of conjugate of leader peptide, D1 AND D2 domains of NKp46 with Fc domain (SEQ ID NO9)
tccccactgctcagcacttaggccggcagaatctgagcgatgtcttccacactccctgccctgctctgcgtcgggctgtgtctgagtcagaggatcagcgcccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatgtatgacacacccaccctctcggttcatcctggacccgaagtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccaggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - DNA, 1110 bp, artificial
  DNA encoding conjugate of CD5 leader peptideand D1 domain of NKP46 with Fc domain (SEQ ID NO10)
aagcttgccgccaccatgggaatgcccatggggtctctgcaaccgctggccaccttgtacctgctggggatgctggtcgcttcctgcctcggacggctcagggtaccccagcagcagactctcccaaaaccgttcatctgggccgagccccatttcatggttccaaaggaaaagcaagtgaccatctgttgccagggaaattatggggctgttgaataccagctgcactttgaaggaagcctttttgccgtggacagaccaaaaccccctgagcggattaacaaagtcaaattctacatcccggacatgaactcccgcatggcagggcaatacagctgcatctatcgggttggggagctctggtcagagcccagcaacttgctggatctggtggtaacagaaatggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 21 - DNA, 1197 bp, artificial
  DNA encoding conjugate of leader peptide and D2 domain of NKP46 with Fc domain (SEQ ID NO12)
aagcttgccgccaccatgggaatgcccatggggtctctgcaaccgctggccaccttgtacctgctggggatgctggtcgcttcctgcctcggacggctcagggtaccctatgacacacccaccctctcggttcatcctggacccgaggtgatctcgggagagaaggtgaccttctactgccgtctagacactgcaacaagcatgttcttactgctcaaggagggaagatccagccacgtacagcgcggatacgggaaggtccaggcggagttccccctgggccctgtgaccacagcccaccgagggacataccgatgttttggctcctataacaaccatgcctggtctttccccagtgagccagtgaagctcctggtcacaggcgacattgagaacaccagccttgcacctgaagaccccacctttcctgacacttggggcacctaccttttaaccacagagacgggactccagaaagaccatgccctctgggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - DNA, 606 bp, homo sapiens <s300> NCBI/BC052582 2004-06-30 (1)..(606)
atggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcctccttcgggctggattctatgctgtcagctttctctctgtggccgtgggcagcaccgtctattaccagggcaaatgtctgacctggaaaggtccaagaaggcagctgccggctgtggtcccagcgcccctcccaccaccatgtgggagctcagcacatctgcttcccccagtcccaggaggctga

SEQ ID NO: 23 - DNA, 1164 bp, artificial
  DNA encoding conjugate of CD5 leader peptide, D1 and D2 domains of NKp30 with Fc domain (SEQ ID NO13)
aagcttgccgccaccatgggaatgcccatggggtctctgcaaccgctggccaccttgtacctgctggggatgctggtcgcttcctgcctcggacggctcagggtacccctctgggtgtcccagccccttgagattcgtaccctggaagggtcttctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 24 - DNA, 854 bp, homo sapiens <s300> NCBI/AJ225109 1999-03-15 (1)..(854)
atggcctggcgagccctacacccactgctactgctgctgctgctgttcccaggctctcaggcacaatccaaggctcaggtacttcaaagtgtggcagggcagacgctaaccgtgagatgccagtacccgcccacgggcagtctctacgagaagaaaggctggtgtaaggaggcttcagcacttgtgtgcatcaggttagtcaccagctccaagcccaggacgatggcttggacctctcgattcacaatctgggacgaccctgatgctggcttcttcactgtcaccatgactgatctgagagaggaagactcaggacattactggtgtagaatctaccgcccttctgacaactctgtctctaagtccgtcagattctatctggtggtatctccagcctctgcctccacacagaccccctggactccccgcgacctggtctcttcacagacccagacccagagctgtgtgcctcccactgcaggagccagacaagcccctgagtctccatctaccatccctgtcccttcacagccacagaactccacgctccgccctggccctgcagcccccattgccctggtgcctgtgttctgtggactcctcgtagccaagagcctggtgctgtcagccctgctcgtctggtggggggacatatggtggaaaaccgtgatggagctcaggagcctggatacccaaaaagccacctgccaccttcaacaggtcacggaccttccctggacctcagtttcctcacctgtagagagagaaatattatatcacactgttgcaaggactaagataagcgatgatgatgatgaacacactttgtgaataataaaattatctgaatgttt

SEQ ID NO: 25 - DNA, 1320 bp, artificial
  DNA encoding conjugate of leader peptide, DS and DL domains of NKp44 with Fc domain (SEQ ID NO15)
aagcttgccgccaccatgggaatgcccatggggtctctgcaaccgctggccaccttgtacctgctggggatgctggtcgcttcctgcctcggacggctcagggtaccccaatccaaggctcaggtacttcaaagtgtggcagggcagacgctaaccgtgagatgccagtacccgcccacgggcagtctctacgagaagaaaggctggtgtaaggaggcttcagcacttgtgtgcatcaggttagtcaccagctccaagcccaggacggtggcttggacctctcgattcacaatctgggacgaccctgatgctggcttcttcactgtcaccatgactgatctgagagaggaagactcaggacattactggtgtagaatctaccgcccttctgacaactctgtctctaagtccgtcagattctatctggtggtatctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagccccggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - DNA, 996 bp, artificial
  DNA encoding conjugate of CD5 leader peptide and DS domain of NKp44 with Fc domain (SEQ ID NO16)
aagcttgccgccaccatgggaatgcccatggggtctctgcaaccgctggccaccttgtacctgctggggatgctggtcgcttcctgcctcggacggctcagggtaccctctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagccccggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 27 - DNA, 1146 bp, artificial
  DNA encoding conjugate of CD5 leader peptide and DL domain of NKp44 with Fc domain (SEQ ID NO17)
aagcttgccgccaccatgggaatgcccatggggtctctgcaaccgctggccaccttgtacctgctggggatgctggtcgcttcctgcctcggacggctcagggtaccccaatccaaggctcaggtacttcaaagtgtggcagggcagacgctaaccgtgagatgccagtacccgcccacgggcagtctctacgagaagaaaggctggtgtaaggaggcttcagcacttgtgtgcatcaggttagtcaccagctccaagcccaggacggtggcttggacctctcgattcacaatctgggacgaccctgatgctggcttcttcactgtcaccatgactgatctgagagaggaagactcaggacattactggtgtagaatctaccgcccttctgacaactctgtctctaagtccgtcagattctatctggtggtatctccagcggatccggagcccaaatcttctgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2007/0280938 A1
(43) Pub. Date: Dec. 6, 2007

(54) Peptides Derived from Natural Cytotoxicity Receptors and Methods of Use Thereof

(75) Inventors: Ofer Mandelboim, Shoham (IL); Angel Porgador, Lehavim (IL)

Correspondence Address: BROWDY AND NEIMARK, P.L.L.C.;624 NINTH STREET, NW, SUITE 300, WASHINGTON, DC, 20001-5303, US

(73) Assignees: YISSUM RESEARCH AND DEVELOPMENT COMPANY OF THE HEBREW UNIVERSITY OF JERUSALEM, 91390 GIVAT RAM, JERUSALEM (IL); BEN-GURION UNIVERSITY OF THE NEGEV RESEARCH AND DEVELOPMENT AUTHORITY, 84105 BEER-SHEVA (IL)

(21) Appl. No.: 10/580,428

(22) PCT Filed: Nov. 24, 2004
(86) PCT No.: PCT/IL04/01081

§ 371 Date: Mar. 15, 2007

Related U.S. Application Data
(60) Provisional application No. 60/524,648, filed on Nov. 25, 2003.

(51) Int. Cl. A61K 39/395; A61K 38/02; A61K 38/10; C07K 16/30; G01N 33/574; C07K 7/08; C07K 14/705; A61K 38/17

(52) U.S. Cl. 424/138.1; 435/7.23; 514/12; 514/13; 514/2; 530/300; 530/324; 530/325; 530/326; 530/350; 530/387.7

(57) ABSTRACT

The present invention relates in general to specific NCR-derived peptides capable of binding to membrane-associated biomolecules of the tumor cells, said biomolecules comprising at least one sulfated polysaccharide. Preferred peptides are about 7 to about 120 amino acids in length and are derived from NKp-44, NKp30 or NKp46.

### FIELD OF THE INVENTION

The present invention relates generally to peptides derived from specific natural cytotoxicity receptors, the peptides capable of binding to membrane-associated biomolecules of tumor and virus infected cells, said biomolecules comprising at least one sulfated polysaccharide, therapeutic compositions comprising the peptides and methods of use thereof.

### BACKGROUND OF THE INVENTION

Natural Killer Cells

Natural Killer (NK) cells are a class of lymphocytes able to destroy virus-infected and transformed cells apparently without prior antigen stimulation (1, 2). The interaction between NK cells and their targets is mediated via a complex array of NK inhibitory and activating receptors (3-7). The ligands of the NK cell surface inhibitory receptors are polymorphic and non-polymorphic major histocompatibility complex (MHC) class I molecules (3-7). Some NK cells express activation receptors specific for MHC class I molecules homologous to various NK inhibitory receptors (3-7).

Three lysis receptors, expressed mainly on human NK cells, have been identified. They are referred to as natural cytotoxicity receptors (NCR) and include the NKp30, NKp44, and NKp46 molecules (3, 5). The NCR are capable of mediating direct killing of tumor and virus-infected cells and are specific for non-MHC ligands. The NCR are highly NK specific, with NKp46 and NKp30 present exclusively on NK cells, whether resting or activated, and NKp44 expressed specifically by activated NK cells (3, 5).

International Patent Publication WO 02/08287 of the present inventors discloses NK receptor fusion proteins in which an extracellular portion of a NK receptor is conjugated to an active segment comprising an immunoglobulin (Ig), a cytotoxic moiety or an imaging moiety. WO 02/08287 further discloses that the NK receptor fusion proteins exhibit specific interaction with tumor cells and viral-infected cells in vitro, and these fusion proteins are disclosed as useful for therapeutic applications in vivo. Specific fusion proteins are disclosed and claimed only for NKp46 and NKp44. The teachings of WO 02/08287 are incorporated herein as if set forth herein in their entirety.

PCT application publication WO 2004/053054 of the present inventors discloses that NK fusion proteins comprising the natural killer cytotoxicity receptor NKp30 or active fragments thereof were found to be especially effective in inhibiting the growth of tumors in vivo. The disclosure further relates to synthetic peptides and fusion proteins comprising NKp30-derived peptide sequences.

PCT application PCT/IL2004/000583 of the present inventors discloses peptides and fusion proteins comprising active glycosylated fragments derived from natural killer cytotoxicity receptors NKp44 and NKp46 that are effective in binding to viral-infected cells and tumor cells. An essential epitope involved in the binding of the NKp46 receptor to viral-infected and tumor cells comprises the threonine 225 (T225) residue, one of the O-glycosylated residues of this molecule. It was further disclosed that a membrane linker peptide derived from the extracellular domain of the human NKp44 receptor is an essential feature in binding to viral infected cells. This linker peptide comprises a hyper-glycosylated region comprising 14 predicted glycosylation sites, which contribute to the efficient binding to viral-infected cells.

PCT publication WO 01/36630 teaches NKp30 specific antibodies that bind to the NKp30 structure, and to the pharmaceutical and therapeutic uses thereof. That application discloses NKp30 polypeptide sequences, including specific peptides comprising amino acids 139-157 and amino acids 157-190, useful as antigens in the production of anti-NKp30 antibodies.

NCR Ligands

The identification of the ligands recognized by the NCR is important for further progress in the NK field. The inventors of the present invention have recently shown that the NKp46 and NKp44 proteins, but not NKp30, recognize hemagglutinin (HA) of influenza virus and hemagglutinin-neuraminidase (HN) of Sendai virus (8-10). The recognition of HA and HN requires the sialylation of NKp46 and NKp44 oligosaccharides. The binding of these NCR to hemagglutinins is required for the lysis of virus-infected cells by NK cells (9, 10).

Previous attempts to identify recognition structures exclusive to the interaction between NK cells and tumor cells have been unsuccessful, although important components on both NK cells and on tumor cells that contribute to cellular adhesion and regulation of the cytolytic process have been revealed. These receptor-ligand interactions, however, are not unique to NK cells since they also occur between T lymphocytes and their respective target cells (11). The surface molecules responsible for NK cell-specific receptor-ligand interactions still remain largely unknown.

Heparan Sulfate Proteoglycans

Membrane-associated heparan sulfate proteoglycans (HISPGs) are known to play important roles in many aspects of cell behavior, including cell-cell and cell-extracellular matrix adhesion and growth factor signaling. Two families of polypeptides appear to carry the majority of the heparan sulfate on mammalian cells: glypicans, which are attached to the plasma membrane via glycosylphosphatidylinositol (GPI) anchors, and syndecans, which are transmembrane proteins. Commonly, cells express multiple HSPGs, from both the glypican and syndecan families.

The role of HSPGs in growth factor signaling has been best characterized with respect to fibroblast growth factors (FGFs), which require the presence of heparan sulfate for high affinity binding to their tyrosine kinase receptors. The requirement of heparan sulfate for FGF signaling is disclosed, for example in U.S. Pat. No. 5,789,182. More recently, several other growth factors have been found to exhibit a strong requirement for an HSPG coreceptor in their signaling (for review see reference 29). These include for example heparin-binding EGF-like growth factor (HB-EGF), hepatocyte growth factor (HGF), and members of the Wnt family of secreted glycoproteins. Many other growth factors, including vascular endothelial growth factor (VEGF), platelet derived growth factor (PDGF), transforming growth factors (TGFs), and bone morphogenetic proteins (BMPs), are known to bind heparin and heparan sulfate, although the physiological consequences of this binding are unclear.

Previous work in tumor cell recognition revealed that membrane-associated heparan sulfate proteoglycans in transformed cells are either over-expressed or modified in their glycosaminoglycan (GAG) content (12-15). For example, glypican 1 which is attached to the plasma membrane via glycosylphosphatidylinositol (GPI) anchors was reported to be overexpressed in breast and pancreatic cancer (12, 13). In another example, aberrant methylation of the heparan sulfate D-glucosaminyl 3-O-sulfotransferase-2 (3-OST-2) gene was found in human breast cancer cells, indicating that silencing of an enzyme associated with the sulfation of heparan sulfate is linked to breast tumors (15).

There exists a long-felt unmet need for identification of the cellular targets of NCR which are responsible for the specific lysis of tumor cells by NK cells. These cellular targets may be used to develop tumor-specific diagnostic, therapeutic and imaging agents.

### SUMMARY OF THE INVENTION

The present invention is based in part on the unexpected discovery that the lysis of tumor cells by NK cells is mediated by the binding of the NK cell, via their natural cytotoxicity receptors (NCRs), to specific sulfated polysaccharide biomolecules of the tumor cells.

In one aspect, the present invention relates to specific isolated NCR-derived peptides capable of binding to membrane-associated biomolecules of the tumor cells, the biomolecules comprising at least one sulfated polysaccharide. According to one embodiment the at least one sulfated polysaccharide is heparan sulfate. Thus, according to one preferred embodiment the peptides of the present invention are capable of binding to heparan sulfate in a tumor cell. These peptides are derived from specific domains of the NCR participating in the interaction with heparan sulfate in the target tumor cell. It is to be clearly understood that the peptides of the invention are smaller than the intact domains of the NCRs from which they are derived.

In one embodiment, the peptides are derived from the D2 domain of NKp46. In one specific embodiment, such peptides comprise the amino acid sequence: FLLLKEGRSSHVQRGYGKVQAEF denoted herein SEQ ID NO: 1, which corresponds to amino acid residues 153-175 of NKp46, or an active fragment, analog or derivative thereof. In one specific embodiment the peptide comprises amino acid sequence: FLLLKEGRSSHVQRGYGKVQ corresponding to amino acids 153-172 (Note: the peptide sequence comprising amino acids 153-172 of NKp46 corresponds to amino acids 132-151 of the receptor given PDB code “loll”. 1 oll is a fragnment of the extracellular region of NKp46, residues 25-212, used in crystallization studies).

In another embodiment, the peptides are derived from NKp30. In one specific embodiment, such peptides comprise the amino acid sequence: RDEVVPGKEVRNGTPEFRGRLAPLASSR denoted herein SEQ ID NO:3, which corresponds to amino acid residues 57-84 of NKp30, or an active fragment, analog or derivative thereof. In another specific embodiment, the peptides comprise the amino acid sequence RDEVVPGKEVRNGTPEFRGR denoted herein as SEQ ID NO:4, which corresponds to amino acid residues 57-76 of NKp30, or an active fragment, analog or derivative thereof.

In yet another embodiment, the peptides are derived from NKp44. In one specific embodiment, such peptides comprise the amino acid sequence: KKGWCKEASALVCIRLVTSSKPRT denoted herein as SEQ ID NO:5, which corresponds to amino acid residues 51-74 of NKp44, or an active fragment, analog or derivative thereof.

The peptides according to the present invention include both linear and cyclic peptides and modified peptides including peptidomimetics, and amidated peptides. A peptide derivative according to the present invention refers to a peptide having various changes, substitutions, insertions, and deletions so long as the peptides retain binding activity. It is to be explicitly understood that the NCRs from which the active fragments are derived, may be of human or non-human origin. Though the human sequences are preferred, non-human primates or even lower mammalian species may be a suitable source for derivation of the active fragments according to the invention. It is further to be explicitly understood that the target cells may be human, as well as non-human mammalian or even avian.

In another aspect, the present invention relates to a method of targeting a tumor cell in a subject in need thereof via an NCR-dependent mechanism, said method comprising administering to the subject an NCR-derived peptide capable of binding to a membrane-associated biomolecule of the tumor cell, the biomolecule comprising at least one sulfated polysaccharide. Accordingly, the present invention relates to the use of an NCR-derived peptide capable of binding to a membrane-associated biomolecule of a tumor cell, the biomolecule comprising at least one sulfated polysaccharide, for the preparation of a medicament useful for targeting a tumor cell.

An NCR derived peptide useful for targeting a tumor cell is useful in the diagnosis, imaging and treatment of benign and malignant tumors and proliferative disease.

Different membrane-associated biomolecules comprising at least one sulfated polysaccharide may serve as a target for the peptides of the present invention. The biomolecules comprising a sulfated polysaccharide include but are not limited to glycosaminoglycans such as heparin, heparan sulfates or dermatan sulfates.

One preferred glycosaminoglycan, which is a binding target for the peptides of the present invention, is heparan sulfate. Other biomolecules comprising a sulfated polysaccharide are glycosaminoglycans covalently attached to proteins such as proteoglycans. Preferred examples of proteoglycans are heparan sulfate proteoglycans (HSPG). HSPG may be divided into two families: glypicans, which are attached to the plasma membrane via glycosylphosphatidylinositol (GPI) anchors, and syndecans, which are transmembrane proteins.

The NCR-derived peptides according to the present invention include active fragments of the NCR but are not limited to a specific size range. However, according to one embodiment of the present invention, the invention provides peptides comprising between about 7 to about 120 amino acid residues in total, preferably between about 8 to about 100 amino acid residues, more preferably the peptides are less than about 50 amino acid residues, preferably about 10 to about 50 amino acids. The present invention also provides peptides in which the core motif sequence is artificially incorporated within a sequence of a polypeptide, including peptides manufactured by recombinant DNA technology or chemical synthesis.

It is to be understood explicitly that the peptides of the present invention are other than full-length NCR polypeptides, linker peptides of the NCR extracellular domains and fragments of an NCR previously disclosed in the art. The present invention excludes specific peptides claimed in WO 02/08287, WO 2004/053054 and PCT application PCT/IL2004/000583.

The NCR-derived peptides of the present invention are capable of binding to specific sulfated polysaccharide biomolecules of the tumor cells. The present invention encompasses NCR-derived peptides incorporated into fusion proteins or conjugated to another molecule or active segment such as immunoglobulin (Ig) or the Fc fragment thereof in order to induce the lysis of tumor cells. Within the scope of the present invention it is contemplated that the binding of the NCR-derived peptides of the present invention may suffice to activate the lysis process in the target tumor cell.

In another aspect, the present invention encompasses antibodies capable of binding to membrane-associated target biomolecules in a tumor cell. In certain embodiments the antibody is capable of mediating NCR-dependent lysis. Such antibodies specifically recognize one or more epitopes present on such target biomolecules mediating the lysis of tumor cells by NK cells via the NCR, said target biomolecules comprising at least one sulfated polysaccharide. According to a specific embodiment, the antibodies bind to a specific heparan sulfate epitope on the target tumor cell, thereby activating the NCR-dependent lysis.

In another aspect, the present invention encompasses specific antibodies capable of blocking the binding of NK cells via their NCR to the membrane-associated target biomolecules in a tumor cell, thereby inhibiting NCR-dependent activity in autoimmunity. A preferred example is an antibody capable of binding to heparan sulfate-associated biomolecules which mediate NCR-dependent lysis.

The present invention also relates to a method for the selective removal of tumor cells from a biological sample which comprises the selective removal of those cells positive for membrane associated biomolecules, the biomolecule comprising at least one sulfated polysaccharide. The method comprises the steps of contacting the biological sample with an antibody of the present invention under conditions appropriate for immune complex formation, and removing the immune complex formed from the biological sample.

The present invention can utilize serum immunoglobulins, polyclonal antibodies or fragments thereof, or monoclonal antibodies or fragments thereof having at least a portion of an antigen binding region, including Fv, F(ab)₂, Fab fragments, single chain antibodies, chimeric or humanized antibodies.

The present invention further relates to a method of targeting a tumor cell in a subject in need, said method comprises administering to the subject an NCR-derived peptide capable of binding to a membrane-associated bio-molecule in the tumor cell, the membrane-associated biomolecule comprising the sulfated polysaccharide according to the present invention.

In the targeting method, preferred NCR-derived peptides according to the present invention are capable of binding to heparan sulfate in a tumor cell. These peptides are derived from specific domains of the NCR participating in the interaction with heparan sulfate in the target tumor cell.

In one embodiment, the peptides derive from the D2 domain of NKp46. In one preferred example, such peptides comprise the amino acid sequence denoted herein SEQ ID NO:1 and SEQ ID NO:2. In another embodiment, the peptides derive from NKp30. In one preferred example, such peptides comprise the amino acid sequence denoted herein SEQ ID NO:3 or SEQ ID NO:4. In yet another embodiment, the peptides derive from NKp44. In one specific embodiment, such peptides comprise the amino acid sequence denoted herein SEQ ID NO:5.

According to yet another aspect, the present invention further relates to pharmaceutical compositions comprising a peptide or a polypeptide of the present invention and a pharmaceutically acceptable carrier. The present invention further encompasses methods of using these compositions for the treatment of malignant and benign tumors including cancer.

The present invention further provides methods of identifying peptides derived from NCR, such peptides capable of targeting tumor cells, by binding to a biomolecule associated with the tumor cells. The present invention further provides methods of identifying peptides derived from NCR, such peptides capable of targeting tumor cells, and mediating lysis upon binding to a biomolecule associated with the tumor cells.

Therefore, according to another aspect the present invention provides a method of identifying peptides derived from NCR which are capable of binding to a biomolecule associated with a tumor cell, the biomolecule comprising at least one sulfated polysaccharide, the method comprising the steps of:
a) providing a set of candidate peptides;
b) contacting the peptides with a tumor cell;
c) determining the binding of said peptides to said tumor cell; and
d) isolating said bound peptides.

These and further embodiments will be apparent from the detailed description and examples that follow.

### BRIEF DESCRIPTION OF THE DRAWINGS

FIG. 1 demonstrates the effect of 6-O-sulfo-LacNAc-PAA on binding of NKp30-Ig and NKp46D2-Ig to tumor cells. (A): Staining of HeLa cells. Results are presented as median fluorescence intensity (MFI). (B) and (C): Staining of HeLa and PC-3 cells, respectively. Results are presented as percentage of binding as compared to staining of cells with NKp46D2-Ig alone without Glyc-PAA mix.

FIG. 2 demonstrates the effect of heparin/heparan sulfate on binding of NKp30-Ig and NKp46D2-Ig to tumor cells. (A): Staining of HeLa cells. Results are presented as MFI; the background staining with CD99-Ig, which does not bind to HeLa cells, was 2 to 3. (B), (C) and (D): Staining of HeLa, PC-3, and HeLa cells, respectively. Results are presented as percentage of binding as compared to staining of cells with NKp46D2-Ig alone, in absence of the GAG or proteoglycan mix.

FIG. 3 demonstrates the effect of heparin/heparan sulfate on binding of NKp44-Ig and NKp44D-Ig to tumor cells. FIGS. 3B and 3C show binding of NKp44-Ig to different GAGS. FIG. 3B shows HeLa cells, FIG. 3C shows PC-3 cells. Results are presented as percentage of binding as compared to staining of cells with NKp44-Ig alone without GAG premix.

FIG. 4 shows the effect of polysaccharide-degrading enzymes and D-mannosamine on binding of NKp30-Ig and NKp46D2-Ig to tumor cells. HeLa (A) and 1106 melanoma (B) cells were incubated in reaction buffer alone (mock treatment) or reaction buffer containing a GAG-degrading enzyme. After incubation cells were washed and stained with fusion-Igs. (C) Staining with NKp46D2-Ig of EB lymphoma, mock-transfected EB and heparanase-transfected EB that express a functional heparanase on the cell surface (EB-SP) (D) Staining with fusion Igs of HeLa cells pretreated with 40mM D-mannosamine overnight. Results are presented as MFI.

FIG. 5 shows the effect of heparin-degrading enzymes, heparan sulfate deficiency and glypican-1 suppression on binding of NKp44-Ig to tumor cells. (A) PC-3 cells were incubated in reaction buffer alone or containing a GAG-degrading enzyme. After incubation, cells were washed and stained with fusion Igs. (B1,2) Staining of parental CHO-K1, heparan sulfate-negative and chondroitin sulfate-negative CHO pgsA-745, and heparan sulfate-negative and chondroitin sulfate high-positive CHO pgsD-677 by NKp44-Ig and human (h) second Ab (primary FACS histogram overlay). (B3, B4) Staining of parental CHO-K1 and CHO pgsA-745 with HS4E4 and mouse (m) second Ab. (C1, C2, C3) Staining of Sham and GAS-6 cells with NKp44-Ig, HS4E4 and hIgG1, respectively (primary FACS histogram overlay). Results are from 1 representative experiment of 2. For all panels, MFI results are the average of 2 different samples assayed in the same experiment. Bars, ±SD.

FIG. 6 shows the effect of 6-O-sulfo-N-acetylglucosamine and target cell-surface heparanase on lysis by primary NK lines. (A) Primary NK line cells were mixed with incremented amounts of Glyc-PAAs and added to Eu-labeled target cells for a 4 h lysis assay. Final concentrations of the disaccharides ranged between 0.225 to 0.9 mM. E:T ration is 50:1. (B) Lysis by primary NK lines of EB, EB-mock transfected and transfected EB-expressing a functional cell-surface heparanase (EB-SP).

FIG. 7 shows that the binding of NKp46 and NKp30 to mutant CHO cells lacking HSPG is significantly reduced as compared to wt CHO cells (A). Panel (B) demonstrates that the lysis of mutant CHO cells lacking HSPG by NK cells is significantly lower than lysis of wt CHO cells.

FIG. 8 shows the electrostatic potential surface of NKp46 (PDB code:1oll). The potential map was calculated and depicted using the program Delphi and Grasp. The surface is marked such that dark areas labeled with an arrow are those having a negative potential (−4 kt/e) unlabeled dark areas are those having a positive potential (+4 kt/e). The positive patch can be seen clearly at the D2 domain of the map. The location of the N-terminus (within the D1 domain) and the C-terminus (within the D2 domain).

FIG. 9 shows superimposition of FN14 and D2 domain of NKp46. Both proteins are depicted in a solid ribbon presentation, for FN14 and NKp46, respectively. Side chains of basic residues associate with HBS-2 in FN14 and the positively charged region in NKp46 are depicted in ball and stick and colored in gray and black respectively. The residue legends corresponds to the color of the side chains.

FIG. 10 shows the binding of NKp46D2-Ig, NKp46D2-Q4-Ig and NKp46D2-Q 4T1-Ig to non-infected and IV-infected cells.

### DETAILED DESCRIPTION OF THE INVENTION

In order that this invention may be better understood, the following terms and definitions are herein provided.

The term “target cells” are cells that are killed by an NCR-dependent mechanism. The target cells express specific sulfated polysaccharide biomolecules and include, in particular, cells that are malignant or otherwise derived from solid as well as non-solid tumors.

The term “NKp46” refers to a natural cytotoxicity receptor expressed on human NK cells that is capable of mediating direct killing of tumor and virus-infected cells. The term “D2 fragment of NKp46” or “NKpD2” refers to domain 2 (the proximal domain) of the NKp46 molecule corresponding to amino acids 121-249 of NKp46.

Heparan sulfate proteoglycans (HSPG) are macromolecules composed of a core protein covalently O-linked to repeating hexuronic and D-glucosamine disaccharide units.

A “glycosaminoglycan” or “GAG” as used herein refers to a long, unbranched polysaccharide molecule found on the cell surface or extracellular matrix. Non-limiting examples of glycosaminoglycans include heparin, chondroitin sulfate, dextran sulfate, dermatan sulfate, heparan sulfate, keratan sulfate, crosslinked or non-crosslinked hyaluronic acid, hexuronyl hexosaminoglycan sulfate, and inositol hexasulfate.

The present invention is based on the first direct proof that saccharides, preferably polysaccharides such as heparan sulfate are potent inhibitors of the binding of NKp30-Ig and NKp46D2-Ig fusion proteins to tumor cells. The structural characteristics of heparin mediating the high affinity binding of NCR to a tumor cell are specific and restricted to highly O-sulfated oligosaccharides. The 6-O-sulfo-N-acetylglucosamine is a building stone of hepariheparan sulfate. It is further disclosed herein that target cell membrane-associated heparan sulfate proteoglycans (HSPGs) are recognized by NKp30-Ig and NKp46D2-Ig. The tumor membrane HSPGs are involved in lysis of target tumor cells by NK cells. Finally, it is now disclosed that the NKp46 three dimensional model revealed the existence of a loop in the second domain of NKp46, between amino acids (aa) 153 to aa 175, with a significant similarity to the heparin binding site 2 (HBS-2) of human fibronectin. Based on a crystal structure of fibronectin type III repeats 12-14 (FN12-14) and biochemical analysis, it is shown that 5 positively-charged amino acids in FN14 (Lys 216, Lys 219, Arg 225, Arg 230 and Lys 261) are critical for fibronectin binding to heparin through HBS-2. The position of the 5 positively-charged aa in the 153-175 loop of NKp46 strikingly overlapped these 5 positively-charged aa of HBS-2.

In one aspect, the present invention relates to specific NCR-derived peptides capable of binding to heparan sulfate in a tumor cell. These peptides are derived from specific domains of the NCR participating in the interaction with heparan sulfate in the target tumor cell.

In one embodiment, the peptides are derived from the D2 domain of NKp46. In one preferred example, such peptides comprise the amino acid sequence denoted as SEQ ID NO:1 or SEQ ID NO:2. In another embodiment, the peptides are derived from NKp30. In one preferred example, such peptides comprise the amino acid sequence denoted as SEQ ID NO:3 or SEQ ID NO:4. In yet another embodiment, the peptides are derived from NKp44. In one preferred example, such peptides comprise the amino acid sequence denoted as SEQ ID NO:5. These peptides are capable of binding to membrane-associated biomolecules in the tumor cells comprising at least one sulfated polysaccharide.

The targeting peptides of the present invention bind to a molecule or structure comprising at least one sulfated polysaccharide that is present preferably only in tumor cells. However, the targeting peptides may bind to a molecule or structure comprising at least one sulfated polysaccharide that is present both in tumor cells and in non-tumor cells. In this case, however, it is preferable that the molecule or structure comprising the sulfated polysaccharides is present in greater amounts in the tumor cells than in the non-tumor cells. Preferably, the molecule or structure comprising the sulfated polysaccharides is present at least at 10-fold higher levels in tumor cells than non-tumor cells. Such molecule or structure may be present at 1000-fold or even higher levels in tumor cells as compared to non-tumor cells.

As disclosed hereinabove, the targeting peptides of the present invention bind to a molecule or structure comprising at least one sulfated polysaccharide. In one embodiment, the molecule or structure comprising the sulfated polysaccharides covalently attached to a protein core. One examples of such sulfated polysaccharides covalently attached to a protein core is the heparan sulfate proteoglycan (HSPG) family of proteins. A few HSPGs were purified to homogeneity, including the large extra-cellular matrix HSPG perlecan, the membrane associated glypicans and the integral membrane syndecans. The syndecans share a similar structure that includes a short highly conserved intracellular carboxy-terminal region, a single membrane-spanning domain and an extracellular domain with three to five possible attachment sites for glycosaminoglycans.

Natural Cytotoxic Receptors

The terms “NKp46”, “NKp30” and “NKp44” refer to the known natural cytotoxicity receptors expressed on NK cells preferably human which is capable of mediating direct killing of tumor and virus-infected cells.

The human NKp46 receptor has multiple isoforms including the currently known isoforms: Isoform a (Accession No CAA04714; SEQ ID NO:6); Isoform b (Accession No. CAA06872; SEQ ID NO:7) Isoform c (Accession No. CAA06873; SEQ ID NO:8) Isoform d (Accession No. CAA06874; SEQ ID NO:9). In general the NKp46 receptor comprises two extracellular Ig-like domains of the C2 type (D1 and D2), a transmembrane portion and an intracellular segment. The extracellular portion of NKp46 comprises a D1 domain, designated NKp46D1 (comprising residues 22-120 of the mature full length protein of isoform a) and a D2 domain, designated NKp46D2, comprising 134 amino acid residues (residues 121-254 of the full length receptor of isoform a).

The human NKp30 receptor (accession number AAH52582; SEQ ID NO:13) comprises one extracellular immunoglobulin (Ig) like domain (residues 31-108)

The human NKp44 receptor (accession number CAB39168 SEQ ID NO:15) comprises one extracellular Ig domain designated herein NKp44D (residues 31-130 of the full length receptor). NKp44DL refers to the Ig-like domain and the NKp44DS refers to the hinge peptide connecting the extracellular domain to the membrane.

The term “cytotoxic effect” refers to a killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell.

The term “specific binding” as used herein refers to the preferential association of a molecule with a cell or tissue bearing a particular target molecule or marker and not to cells or tissues lacking that target molecule or expressing that target molecule at low levels. It is, of course, recognized that a certain degree of non-specific interaction may occur between a molecule and a non-target cell or tissue.

The term “conjugate” refers to a polypeptide formed by the joining of two or more polypeptides through a peptide bond formed between the amino terminus of one polypeptide and the carboxyl terminus of another polypeptide. The conjugate may be formed by the chemical coupling of the constituent polypeptides or it may be expressed as a single polypeptide fusion protein from a nucleic acid (polynucleotide) sequence encoding the single contiguous conjugate.

The term “active fragments” refers to “fragments”, “variants”, “analogs” or “derivatives” of the molecule. A “fragment” of a molecule, such as any of the nucleic acid or the amino acid sequence of the present invention is meant to refer to any nucleotide or amino acid subset of the molecule. A “variant” of such molecule is meant to refer to a naturally occurring molecule substantially similar to either the entire molecule or a fragment thereof. An “analog” of a molecule is a homologous molecule from the same species or from different species. The amino acid sequence of an analog or derivative may differ from the specific molecule, e.g. the NKp46, NKp3O or NKp44 receptors, used in the present invention when at least one residue is deleted, inserted or substituted.

The term “cellular ligand” refers generally to tumor cell membrane molecules capable of reacting with the target recognition segment of the peptide of the invention.

The term “target cells” refers to cells that are killed by the cytotoxic activity of the peptide of the invention. The target cells express the ligand for at least one of NKp46, NKp30 and NKp44 molecules and include, in particular, cells that are infected by a virus, cells-that are malignant or other wise derived from solid as well as non-solid tumors. The target cell is of mammalian origin.

The term “cell-mediated cytotoxicity or destruction” refers to antibody-dependent, cell-mediated cytotoxicity (ADCC) and natural killer (NK) cell killing.

Peptides, Pepitidomimetics and Peptide Derivatives

Within the scope of the invention are included peptides, peptidomimetic and peptide analogs and peptide derivatives. The peptides to be used in the present invention may be prepared for example by the F-moc technique (52), or any other method of peptide synthesis known to those skilled in the art, such as for example by solid phase peptide synthesis. These fragments could also be produced by methods well known to one skilled in the art of biotechnology. For example, using a nucleic acid selected from the group including DNA, RNA, or cDNA. The desired fragments may be produced in live cell cultures and purified after cell harvesting as known in the art.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The present invention further comprises peptide derivatives and peptidomimetics. A peptide mimetic or peptidomimetic, is a molecule that mimics the biological activity of a peptide but is not completely peptidic in nature. Whether completely or partially non-peptide, peptidomimetics according to this invention provide a spatial arrangement of chemical moieties that closely resembles the three-dimensional arrangement of groups in the peptide on which the peptidomimetic is based. As a result of this similar active-site geometry, the peptidomimetic has effects on biological systems which are similar to the biological activity of the peptide.

Without wishing to be bound by theory, the present invention encompasses peptide, peptide analog and peptidomimetic compositions, in which the peptide, peptide analog and peptidomimetic are capable of binding to a membrane associated biomolecule of a tumor cell, the biomolecule comprising at least one sulfated polysaccharide. Said peptide/peptidomimetic composition contributes to the treatment of any tumor cell, including solid and non-solid tumor cells. Said peptide/peptidomimetic compositions are effective in situations where targeting and lysing of a tumor cell is beneficial, including but not limited to proliferative diseases such as carcinomas of various tissues, melanomas, gliomas, lymphomas and the like.

Another aspect of the present invention encompasses peptide/peptidomimetic compositions capable of inhibiting tumor cell progression and proliferation.

There are clear advantages for using a mimetic of a given peptide rather than the peptide itself, because peptides commonly exhibit two undesirable properties: poor bioavailability and short duration of action. Peptide mimetics offer a route around these two major obstacles, since the molecules concerned have a long duration of action. Furthermore there are problems associated with stability, storage and immunoreactivity for peptides that are not experienced with peptide mimetics.

The design of the peptidomimetics may be based on the three-dimensional structure of the extracellular domain of NCR with or in complex with their ligands. Peptidomimetics are small molecules that can bind to ligands such as proteins and glycosaminoglycans by mimicking certain structural aspects of peptides and proteins. A primary goal in the design of peptide mimetics has been to reduce the susceptibility of mimics to cleavage and inactivation by peptidases, as described supra. Some techniques for preparing peptidomimetics are disclosed in U.S. Pat. Nos. 5,550,251 and 5,288,707, for example. Non-limiting examples of the use of peptidomimetics in the art include anti-cancer drugs (U.S. Pat. No. 5,965,539) inhibitors of p21 ras (U.S. Pat. No. 5,910,478) and inhibitors of neurotropin activity (U.S. Pat. No. 6,291,247).

As contemplated by this invention, the term “peptide” includes modified forms of the peptide, so long as the modification does not alter the essential sequence and the modified peptide retains the ability to bind to a membrane-associated biomolecule of a tumor cell. Such modifications include amidation, N-terminal acetylation, glycosylation, biotinylation, etc. Particular modified versions of the L-amino acid peptides corresponding to the amino acid sequences SEQ ID NOS:1-5are described below and are considered to be peptides according to this invention:
a) Peptides with an N-Terminal D-Amino Acid: The presence of an N-terminal D-amino acid increases the serum stability of a peptide which otherwise contains L-amino acids, because exopeptidases acting on the N-terminal residue cannot utilize a D-amino acid as a substrate. The amino acid sequences of the peptides with N-terminal D-amino acids are usually identical to the sequences of the amino acid peptides described above [e.g., SEQ ID NO:1-5], except that the N-terminal residue is a D-amino acid.
b) Peptides with a C-Terminal D-Amino Acid: The presence of a C-terminal D-amino acid also stabilizes a peptide, which otherwise contains L-amino acids, for the same reason as in (a) above. Thus, the amino acid sequences of these peptides are usually identical to the sequences of the L-amino acid peptides described above [e.g., SEQ ID NO:1-5] except that the C-terminal residue is a D-amino acid.
c) Cyclic Peptides: Cyclic peptides have no free N- or C-termini. Thus, they are not susceptible to proteolysis by exopeptidases, although they may be susceptible to endopeptidases, which do not cleave at peptide termini. The amino acid sequences of the cyclic peptides may be identical to the sequences of the L-amino acid peptides described above except that the topology is circular, rather than linear.
d) Peptides with Substitution of Natural Amino Acids by Unnatural Amino Acids: Substitution of unnatural amino acids for natural amino acids can also confer resistance to proteolysis. Such a substitution can, for example, confer resistance to proteolysis by exopeptidases acting on the N-terminus. Such substitutions have been described (53) and these substitutions do not affect biological activity. Furthermore, the synthesis of peptides with unnatural amino acids is routine and known in the art (53).
E. Peptides with N-Terminal or C-Terminal Chemical Groups: An effective approach to confer resistance to peptidases acting on the N-terminal or C-terminal residues of a peptide is to add chemical groups at the peptide termini, such that the modified peptide is no longer a substrate for the peptidase. One-such chemical modification is glycosylation of the peptides at either or both termini. Certain chemical modifications, in particular N-terminal glycosylation, have been shown to increase the stability of peptides in human serum (54). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group. Synthesis of N-substituted oligomers is disclosed in U.S. Pat. No. 5,877,278.
F. Peptides with Additional Amino Acids: Also included within this invention are modified peptides which contain within their sequences the peptides described above. These longer peptide sequences, which result from the addition of extra amino acid residues are encompassed in this invention, since they have the same biological activity as the peptides described above.

Based on the available amino acid sequence of the extracellular domains of the different NCR, presented herein, the three-dimensional structure models of NKp46, NKp30, and NKp44 from X-ray crystal structure, commercially available software packages can be used to design small peptides and/or peptidomimetics, preferably non-hydrolyzable analogs, as specific antagonists/inhibitors.

Suitable commercially available software for analyzing crystal structure, designing and optimizing small peptides and peptidomimetics are well known to one with skill in the art.

The peptides of the present invention are peptides or peptide analogs having amino acid sequence derived from the NCR and peptidomimetics based on the structure of such peptides.

A preferred embodiment of the present invention is a peptidomimetic or a peptide fragment including 7 to 120 consecutive residues, preferably about 8 to about 100 residues, more preferably about 10 to about 50 residues having a sequence derived from the extracellular domain of the NCR encompassing the biomolecule binding site of the receptor. For NKp46, the currently preferred embodiments comprise a sequence derived from residues 153-175 (SEQ ID NO:1), or from residues 153-172 (SEQ ID NO:2) wherein the biomolecule comprises at least one sulfated polysaccharide. For other NCR preferred embodiments comprise an about 7 to about 120 residue peptidomimetic or a peptide fragment derived from: NKp30 residues 57-84 (SEQ ID NO: 3); NKp30 residues 57-76 (SEQ ID NO:4); NKp44 residues 51-74 (SEQ ID NO:5). (The amino acid residues according to the polypeptides that include the leader peptide).

The present invention further provides a method of identifying peptides derived from NCR which are capable of binding to a membrane-associated sulfated polysaccharide of a tumor cell, comprising the steps of:
a. providing a set of candidate peptides;
b. contacting the peptides with the tumor cell;
c. determining the binding of said peptides to said tumor cell; and
d. isolating said bound peptides.

Candidate peptides may be selected stochastically from the sequence of the NCRs or using bioinformatics and or modeling techniques. The candidate peptides are generally prepared by recombinant methods or by peptide synthesis methods known in the art. Peptides of about 7 to about 120 amino acids are preferred. In one embodiment, the peptide is labeled with a reporter enzyme, isotopic label or fluorescence label. Binding of the peptides to the tumor cells and detection of binding may be performed by methods known in the art including, in a non-limiting example, direct and indirect methods such as ELISA.

Antibodies

The present invention further relates to an isolated antibody, preferably a monoclonal antibody which specifically binds to a molecule or structure comprising at least one sulfated polysaccharide in tumor cells and thus activates the NCR-dependent lysis of tumor cells. The isolated antibody of the invention can be coupled to any appropriate label for visualization purposes. Such labels include e.g. fluorescent labels, radioactive labels, enzymatic labels. The antibodies of the present invention are useful in diagnostic, therapeutic and imaging methods.

In another aspect, the present invention encompasses specific antibodies capable of blocking the binding of NK cells via their NCR to the membrane-associated target biomolecules in a tumor cell, thereby inhibiting NCR-dependent activity in autoimmunity. A preferred example is an antibody capable of binding to heparan sulfate-associated biomolecules which mediate NCR-dependent lysis.

The monoclonal antibodies (mAb) of the invention can be prepared using any technique that provides for the production of antibody molecules by cell lines in culture. These include, but are not limited to, the original techniques of Köhler and Milstein, (55), modified as described in (56), the contents of which are hereby incorporated by reference.

Screening procedures that can be used to screen hybridoma cells producing antibodies, but are not limited to (1) enzyme-linked immunoadsorbent assays (ELISA), (2) immunoprecipitation or (3) fluorescent activated cell sorting (FACS) analyses. Many different types of ELISA that can be used to screen for the monoclonal antibodies can be envisioned by persons skilled in the art.

Once the desired hybridoma has been selected and cloned, the resultant antibody may be produced in one of two major ways. The purest monoclonal antibody is produced by in vitro culturing of the desired hybridoma in a suitable medium for a suitable length of time, followed by the recovery of the desired antibody from the supernatant. The length of time and medium are known or can readily be determined. This in vitro technique produces essentially monospecific monoclonal antibody, essentially free from other species of anti-human immunoglobulin. However, the in vitro method may not produce a sufficient quantity or concentration of antibody for some purposes, since the quantity of antibody generated is only about 50 μg/ml. To produce a much larger quantity of monoclonal antibody, the desired hybridoma may be injected into an animal, such as a mouse. Preferably the mice are syngeneic or semi-syngeneic to the strain from which the monoclonal-antibody producing hybridomas were obtained. Injection of the hybridoma causes formation of antibody producing tumors after a suitable incubation time, which will result in a high concentration of the desired antibody (about 5-20 mg/ml) in the ascites of the host animal.

Antibody molecules can be purified by known techniques e.g. by immunoabsorption or immunoaffinity chromatography, chromatographic methods such as high performance liquid chromatography or a combination thereof.

In another aspect, the invention relates to isolated immuno-reactive fragments of the antibody of the invention. Such fragments notably include Fab, F(ab′)₂, and CDR antibody fragments. The skilled person will note that humanized antibodies of the invention can be derived therefrom as desired, notably when intended to be administered to a human person. By “immuno-reactive fragments of an antibody”, it is meant any antibody fragment comprising the antigen binding-site.

Such fragments thus include F(ab′)₂ fragments obtained either by enzymatic digestion of said antibody by proteolytic enzymes such as pepsin or papain, and Fab fragments derived thereof by reduction of the sulfhydryl groups located in the hinge regions, as known by any skilled person. Immunoreactive fragments can also comprise recombinant single chain or dimeric polypeptides whose sequence comprises the CDR regions of the antibody of interest. Isolated CDR regions themselves are also contemplated within the definition of the isolated immuno-reactive fragments of the invention.

Chimeric antibodies are molecules, the different portions of which are derived from different animal species, such as those having a variable region derived from a murine mAb and a human immunoglobulin constant region. Antibodies which have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Better et al, 1988; Cabilly et al, 1984; Harlow et al, 1988; Liu et al, 1987; Morrison et al, 1984; Boulianne et al, 1984; Neuberger et al, 1985; Sahagan et al, 1986; Sun et al, 1987; PCT patent applications WO 86/01533, WO 97/02671, WO 90/07861, WO 92/22653 and U.S. Pat. Nos. 5,693,762, 5,693,761, 5,585,089, 5,530,101 and 5,225,539). These references are hereby incorporated by reference.

In addition to the conventional method of raising antibodies in vivo, antibodies can be generated in vitro using phage display technology. Such a production of recombinant antibodies is much faster compared to conventional antibody production and they can be generated against an enormous number of antigens. In contrast, in the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies one can use various methods all based on phage display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

The present invention also relates to a method for the selective removal of tumor cells from a biological sample, which comprises the selective removal of those cells having at least one membrane-associated biomolecules comprising at least one sulfated polysaccharide. Such a method comprises contacting the biological sample with the isolated antibody of the present invention or the immunoreactive fragments thereof under condition appropriate for immune complex formation, and removing the immune complex thus formed.

According to various embodiments, a biological sample includes peripheral blood, plasma, bone marrow aspirates, lymphoid tissues, as well as cells isolated from cytapheresis, plasmapheresis and collection fluids such as synovial, cerebro-spinal, broncho-alveolar and peritoneal fluids.

Pharmaceutical Compositions and Pharmacokinetics

The present invention is also directed to pharmaceutical compositions comprising a pharmaceutically acceptable carrier and at least one peptide, peptide derivative or peptidomimetic of the present invention. The pharmaceutical composition will be administered according to known modes of peptide administration, including oral, intravenous, subcutaneous, intraarticular, intramuscular, inhalation, intranasal, intrathecal, intradermal, transdermal or other known routes. The dosage administered will be dependent upon the age, sex, health condition and weight of the recipient, and the nature of the effect desired.

The composition of the invention further comprises a pharmaceutically acceptable diluent or carrier. The compositions according to the invention will in practice normally be administered orally or by injection. As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

For oral administration tablets and capsules may contain conventional excipients, such as binders, for example syrup, sorbitol, or polyvinyl pyrrolidone; fillers, for example lactose, microcrystalline cellulose, corn starch, calcium phosphate or sorbitol; lubricants, for example magnesium stearate, stearic acid, polyethylene glycol or silica; disintegrates, for example potato starch or sodium starch glycolate, or surfactants, such as sodium lauryl sulphate. Oral liquid preparations can be in the form of for example water or oil suspensions, solutions, emulsions, syrups or elixirs, or can be supplied as a dry product for constitution with water or another suitable vehicle before use. A composition according to the invention can be formulated for parenteral administration by injection or continuous infusion. Compositions for injection can be provided in unit dose form and can take a form such as suspension, solution or emulsion in oil or aqueous carriers and can contain formulating agents, such as suspending, stabilizing and/or dispersing agents. Alternatively, the active constituent can be present in powder form for constitution with a suitable carrier, for example sterile pyrogen-free water, before use. Intravenous administration, for example, is advantageous in the treatment of leukemias, lymphomas, and comparable malignancies of the lymphatic system. The composition of the invention may be administrated directly into a body cavity adjacent to the location of a solid tumor, such as the intraperitoneal. cavity, or injected directly into or adjacent to a solid tumor.

Methods of Treatment

It is proposed that the various methods and compositions of the invention will be broadly applicable to the treatment of any tumor cell, including solid and non-solid tumor cells. Further provided is use of the compositions of the invention for the preparation of a medicament for the treatment of tumor cells and proliferative diseases. If the tissue is a part of the lymphatic or immune systems, malignant cells may include non-solid tumors of circulating cells. Malignancies of other tissues or organs may produce solid tumors. Exemplary solid tumors to which the present invention is directed include but are not limited to carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, neuroblastomas, and the like. Exemplary non-solid tumors to which the present invention is directed include but are not limited to B cell Lymphoma, T cell Lymphoma, or Leukemia such as Chronic Myelogenous Leukemia.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

Cell Lines: The cell lines used herein were as follows:

PC-3: a human prostate adenocarcinoma derived from bone metastasis that is PSA negative and Androgen insensitive (ATCC no. CRL-1435).

1106: a human melanoma cell line that expresses no HLA-I antigens, established from a recurrent metastatic lesion (30).

HeLa: a human cervical adenocarcinoma (ATCC no. CCL-2).

EB-SP: EB murine T-lymphoma transfected with cDNA encoding for chimeric functional heparanase comprising human and chicken heparanase signal peptides (16).

PANC-1: human pancreatic ductal carcinoma (ATCC no. CRL-1469) over-expressing glypican-1 (31).

GAS6: PANC-1 cells stably transfected with full-length glypican-1 antisense construct, having reduced glypican-1 expression at both the RNA and protein levels;

Sham-PANC-1: control-transfected PANC-1 cells, high levels of glypican-1 (31).

Wild type CHO K1 cells and the mutant derivatives CHO pgsA-745 and CHO pgsD-677 were kindly supplied by Dr. Jeff Esko (32).

NK cells (lines and clones) were isolated from peripheral blood lymphocytes (PBL) using the human NK cell isolation kit and the autoMACS instrument (Miltenyi Biotec GmbH, Bergisch Gladbach, Germany). The NK cells were kept in culture as previously described (33).

Carbohydrates and Proteoglycans

Glyc-PAAs are carbohydrate complexes in which Glyc is the oligosaccharide part and PAA is a soluble polyacrylamide carrier of 30 kDa. The content of oligosaccharides in the conjugates is 20% mol. Thus, for LacNAc-PAA, on the average, each fifth unit of the PAA polymer is conjugated to LacNAc and the oligosaccharide content is 1.05 μmol LacNAc/mg Glyc-PAA (17). A library of 35 different Glyc-PAAs containing carbohydrate ligands for siglecs, galectins, selectins and others was used for initial screen and further identification. Low molecular weight (LMW) Heparin (H-3400), Heparan sulfate (H-9902), Hyaluronic acid (H-5388), Chondroitin sulfate A (C-8529) and Chondroitin sulfate C (C-4384) were purchased from Sigma (St. Louis, Mo.; 10 mg/ml). High molecular weight heparins that are modified in N-sulfation, O-sulfation and acetylation were described (18).

Ig-Fusion Proteins

The generation of NKp30-Ig, NKp46-Ig, CD99-Ig and LIR1-Ig fusion protein was previously described (32, 19, 10). To generate the NKp46D2-Ig truncated fusion protein in COS cells, residues 101-235 (D2) of the mature NKp46 protein were PCR amplified, and the PCR-generated fragment was cloned into a mammalian expression vector containing the Fc portion of human IgG1, as previously described (34). In order to allow expression of NKp46D2-Ig, which lacks its original leader peptide sequence, a methionine start codon was added and cloned in tandem to the PCR-amplified fragment of NKp46D2 and in frame with the leader peptide of the CD5 antigen (accession number NP_{—}055022).

The sequences for the truncated fusion proteins, NKp44D-Ig (residues 1-111) was amplified by PCR from the NKp44-Ig-encoding plasmid and the corresponding PCR fragments, containing kozak sequence and leader sequence of CD5, were cloned back into the pcDNA 3.1-Ig vector. Sequencing of the construct revealed that all cDNAs were in frame with the human Fc genomic DNA and were identical to the reported sequences. COS-7 cells were transiently transfected with the construct using FuGENE6® reagent (Roche Molecular Biochemicals, Indianapolis, Ind.) according to the manufacturer's instructions, and supernatants were collected and purified on a protein G column. SDS-PAGE analysis revealed that all Ig fusion proteins were approximately 95% pure and had the proper molecular mass.

For the production of NKp30-Ig and NKp46D2-Ig in CHO cells, the corresponding PCR fragments containing kozak sequence and leader sequence of CD5 were cloned into pcDNA 3.1-Ig vector. CHO cells were transfected with these expression vectors and G418-selected clones were screened for highest protein production. Re-cloned high producer clones were grown in CHO-SFM II medium (Gibco-BRL, Paisley, UK) and supernatants were collected daily and purified on protein-G columns using FPLC.

The conjugated proteins and their corresponding polynucleotides are referred to herein as follows:

SEQ ID NO:10 protein conjugate comprising NKp46 D1 and D2 domains fused to the Fc domain of an Ig;

SEQ ID NO:20 DNA encoding protein conjugate having SEQ ID NO:10;

SEQ ID NO:11 protein conjugate comprising CD5 leader sequence and NKp46 D1 domain fused to Fc domain;

SEQ ID NO:21 DNA encoding protein conjugate having SEQ ID NO: 11;

SEQ ID NO:12 protein conjugate comprising CD5 leader sequence and NKp46 D2 domain fused to Fc domain;

SEQ ID NO:22 DNA encoding protein conjugate having SEQ ID NO:12;

SEQ ID NO:14 protein conjugate comprising CD5 leader sequence and NKp30 D domain fused to Fc domain;

SEQ ID NO:24 DNA encoding protein conjugate having SEQ ID NO:14;

SEQ ID NO:16 protein conjugate comprising CD5 leader sequence and NKp44 DS and DL domains fused to Fc domain;

SEQ ID NO:26 DNA encoding protein conjugate having SEQ ID NO:16;

SEQ ID NO:17 protein conjugate comprising CD5 leader sequence and NKp44 DL domain fused to Fc domain;

SEQ ID NO:27 DNA encoding protein conjugate having SEQ ID NO: 17;

SEQ ID NO:18 protein conjugate comprising CD5 leader sequence and NKp44 DS domain fused to Fc domain;

SEQ ID NO:28 DNA encoding protein conjugate having SEQ ID NO: 18.

Flow Cytometry and Antibodies

Cells were incubated with the various fusion-Igs for 2 h at 4° C., washed and stained with FITC-conjugated-F(ab′)₂-Goat-anti-human-IgG-Fcy (109-096-098, Jackson ImmunoResearch, West Grove, Pa.). Staining and washing buffer consisted of 0.5% (w/v) BSA and 0.05% sodium azide in PBS. Staining of CHO and mutant CHO cells was carried out with 2% FCS instead of bovine serum albumin (BSA) in the different buffers. Propidium iodide (PI) was added prior to reading for exclusion of dead cells. Flow cytometry was performed using a FACSCalibur flow cytometer (Becton Dickinson, Mountain View, Calif.). Data files were acquired and analyzed using BD CELLQuest™ 3.3 software.

For most binding inhibition experiments, 20 μg of fusion-Ig were premixed with the GAG and added to cells for staining as above. In all experiments, each sample was stained twice in different wells. When results are presented as MFI (median fluorescence intensity), average MFI∓SD of the duplicate staining is shown to reveal consistency of staining procedure. Human IgG1 (hIgG1 kappa, PHP010) was purchased from Serotec, Oxford, UK. Staining with the anti-heparin/heparan sulfate antibody HS4E4 was previously described (35).

Glycosidases and Treatment of Cells

Tumor cells (10⁶) were washed twice in PBS, resuspended in 1ml reaction buffer alone (mock) or reaction buffer containing one of the following GAG-degrading enzymes (Sigma): keratanase (0.94 u/ml, K-2876), heparin lyase I (1.56 u/ml, H-2519) and heparin lyase III (1.25 u/ml, H-8891). Reaction buffer consisted of 1% (w/v) BSA, 1 μg/ml leupeptin and 10 u/ml aprotinin in PBS. Cells were incubated with enzyme for 60 min at 37° C., washed two times with PBS and stained with fusion-Igs as above.

Cytotoxicity Assays

The cytotoxic activity of NK lines against the various targets was assessed in 5-hr 35S-release assays and in 4-hr Eu release time-resolved fluorescence assays, as previously described (20). In experiments where carbohydrates were included, NK cells were first mixed with the carbohydrates and then added to target cells. In all experiments shown the spontaneous release was less than 25% of maximal release. Each point represents the average of duplicate/triplicate values. The range of the duplicates/triplicates was within 5% of their mean.

### Example 1

### Binding of NKp6D2-Ig and NKp30-Ig to Tumor Cells is Inhibited by N-acetylglucosamine

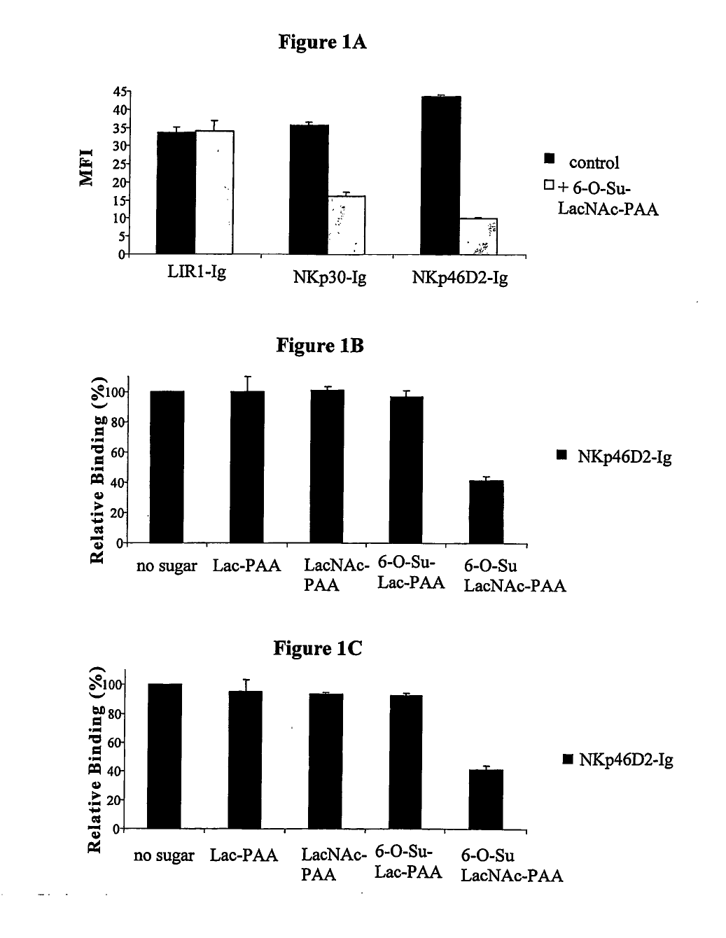

To study the effect of glycosylation on the binding of NKp46D2-Ig and NKp30-Ig to tumor cells, a library of 35 different polyacrylamide-glycoconjugates (Glyc-PAAs) containing carbohydrate ligands for siglecs, galectins, selectins and others were screened. Glyc-PAAs were mixed with Ig-fusion proteins, and staining of tumor cells with the Ig-fusion protein was measured. One Glyc-PAA, in which the saccharide moiety was 6-O-sulfo-LacNAc, inhibited binding of NKp46D2-Ig to HeLa cells (FIG. 1A). Similarly, the binding of NKp30-Ig was inhibited but not the positive binding of LIR1-Ig (FIG. 1A). Similar phenotype was observed when other tumor cell lines, 1106 melanoma and PC-3 prostate cancer, were assessed (FIG. 1C). Pre-incubation of the cells with 6-O-sulfo-LacNAc-PAA, followed by wash and application of the fusion proteins did not affect the binding. The contribution of the different glucose modifications to the inhibition of binding was furter analyzed. FIGS. 1B, C show that removal of either the sulfate, acetyl or both from the N-acetylglucosamine abolished the effect on binding of NKp46D2-Ig to HeLa And PC-3 cells. Similar results were obtained for NKp30-Ig.

The effect of sulfation of galactose in LacNAc-PAA on the binding of Ig-fusion proteins was further studied. 3′-O-sulfo-LacNAc and 4 ′, 6′-di-O-sulfo-LacNAc did not inhibit binding of NKp46D2-Ig or NKp30-Ig, while 6′-O-sulfo-LacNAc manifested inconsistent inhibition phenotype of up to 25% reduction in binding.

To summarize, cell membrane-associated oligosaccharides appear to be involved in the binding of NKp46D2-Ig and NKp30-lg to their cellular ligands, and 60-sulfo-N-acetylglucoamine appears be one of the building stones of these oligosaccharides.

### Example 2

### Binding of NCR-Igs to Tumor Cells is Inhibited by Heparin/Heparan Sulfate: O-Sulfation and Acetylation are Involved

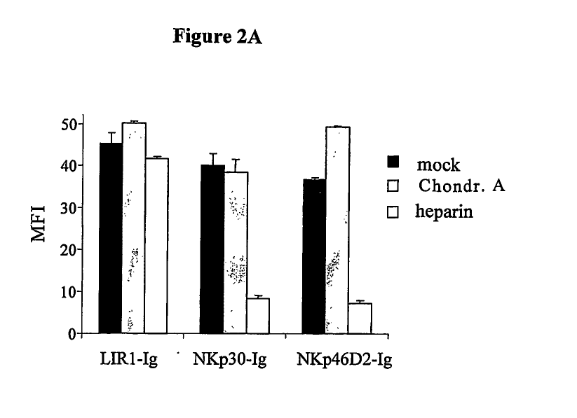

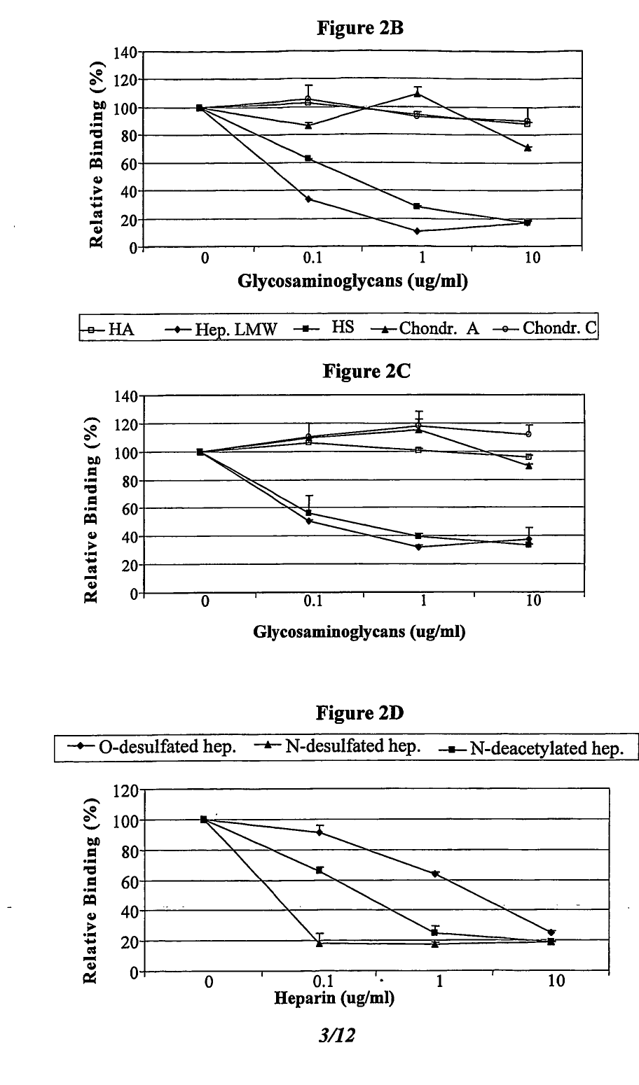

The nature of the sulfated saccharide involved in binding of NKp46D2-Ig and NKp30-Ig to tumor cells was further evaluated by determining the possible role for glycosaminoglycans (GAGs). HeLa cells were incubated with mix of low molecular weight (LMW) heparin (10 μg/ml; white bar)) and either NKp46D2-Ig, NKp30-Ig or LIR1-Ig. All 3 fusion proteins bound well to HeLa cells and heparin inhibited the binding of NKp46D2-Ig and NKp30-Ig, but not the binding of LIR1-Ig (FIG. 2A). Chondroitin A (gray bar/did not inhibit the binding of either of the 3 fusion proteins (FIG. 2A). Pre-incubation of the cells with LMW heparin, followed by wash and application of the fusion proteins did not affect the binding (data not shown).

The specific role of heparin/heparan sulfate in inhibition of NKp46D2-Ig binding to HeLa and PC-3 tumor cells is further shown in FIGS. 2B and 2C. Incremental concentrations of chondroitin A, chondroitin C and hyaluronic acid up to 10 μg/ml did not inhibit binding of NKp46D2-Ig. In contrast, heparin LMW and heparan sulfate in concentrations of 0.1 μg/ml inhibit binding of these fusion proteins. Similar results were obtained for NKp30-Ig.

The influence of variations in sulfation and acetylation of heparin on its capacity to inhibit the binding of NKp46D2-Ig and NKp30-Ig to tumor cells was examined. N-desulfation of heparin resulted in the removal 100% of the N-sulfate groups while O-desulfation removed 99% of the O-linked sulfates (18). N-desulfated heparin was a potent inhibitor of NKp46D2-Ig binding while O-desulfation of heparin reduced significantly the observed inhibition (FIG. 2D). Deacetylated heparin in which all N-acetyl groups were replaced by N-hexanoyl was then tested. This modification also reduced the potential of the heparin to inhibit binding of NKp46D2-Ig (FIG. 2D). Similar results were obtained for NKp30-Ig.

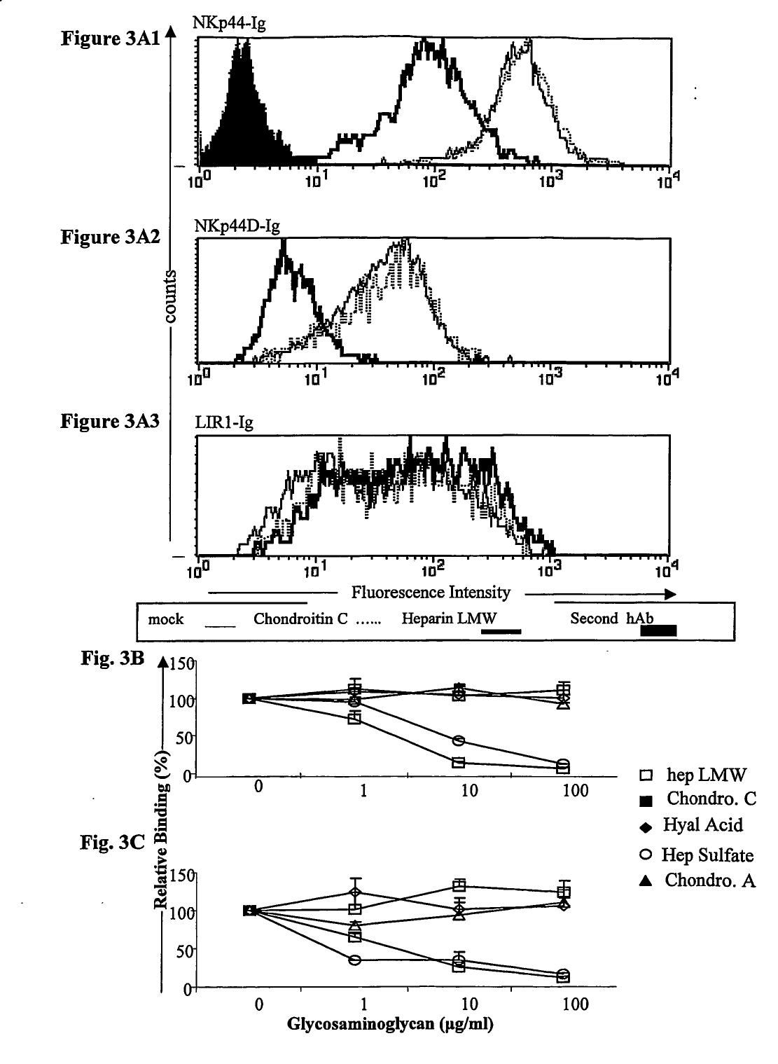

Similar inhibition results were shown for the heparin/heparan sulfate dependent binding of NKp44-Ig and NKp44D-Ig to tumor cells (FIGS. 3A-3C). Twenty μg fusion-Ig were premixed with different GAGs and 10⁵ cells were then added for 2 h, 4° C. The heparan sulfate concentration was approximately 0.3 μM. After incubation, cells were washed and incubated with FITC-anti-Fc second antibody. PI was added to exclude dead cells. FIGS. 3A1, A2 and A3 show staining of HeLa cells with NKp44-Ig, NKp44D-Ig and LIR1-Ig, respectively (primary FACS histogram overlays).

FIGS. 3B and 3C show the effect of titrated concentration of different GAGs (10 ug/ml; oLMW heparin; ▪chondroitin C; ♦hyaluronic acid; oheparin sulfate; ▴chondroitin A) on NKp44-Ig binding. Binding to HeLa (FIG. 3B) and PC-3 (FIG. 3C). Results are presented as percentage of binding as compared to binding to cells with NKp44-Ig alone without premixing with a GAG. Results are from one representative experiment of two performed. In panels 3B and 3C, results are the average of 2 samples assayed in the same experiment. Bars, ±SD.

### Example 3

### NCR-Igs Bind to Heparan Sulfate on Tumor Cells

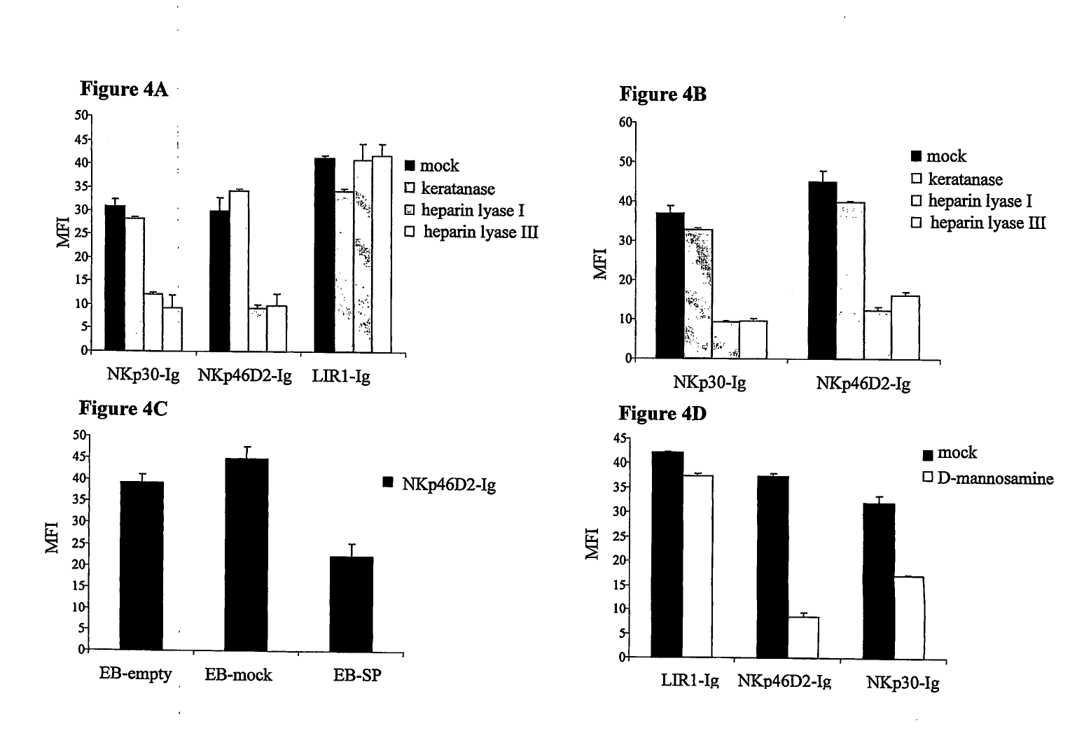

The involvement of cell membrane-associated GAGs in the binding of NKp46D2-Ig and NKp30-Ig to their cellular ligands was examined. 6-O-sulfo-N-acetylglucosamine is a component of keratan sulfate and heparin/heparan sulfate but not of chondroitin sulfate and dermatan sulfate. Therefore, tumor cells were treated with (i) heparin lyase III (white bars) that efficiently degrades heparan sulfate and with a broad specificity, and (ii) heparin lyase I (light gray bars) that is selective in cleaving highly sulfated heparan sulfate. Yet, they do not degrade keratan sulfate and chondroitin sulfates A-E (21). Tumor cells were also treated with keratanase (dark gray bars) that efficiently degrade keratan sulfate but not other GAGs. Treatment of HeLa and 1106 melanoma cells with heparin lyase I or III, but not with keratanase, reduced the binding of NKp46D2-Ig and NKp3O-Ig by 60 to 70% (FIG. 4A, 4B). LIR1-Ig did not bind to 1106 melanoma cells, thus the specificity of heparin lyase treatment on binding of LIR1-Ig to HeLa cells was studied. Both heparin lyase I and III treatments did not reduce the binding of LIRI-Ig to HeLa cells (FIG. 4A). Heparan sulfates are attached to the core protein primarily by O-linked glycoside bonds while keratan sulfates are attached primarily by N-linked bonds (22). In accordance, treatment of tumor cells with a blocker of O-glycosylation, abenzyl-GalNAc, significantly reduced the binding of NKp30-Ig and NKp46D2-Ig.

The EB T lymphoma cell line and EB-SP cells that express a functional heparanase on the cell surface (16) were stained with NKp46D2-Ig. Staining of EB-SP was reduced by 50% as compared to parental EB or EB-mock transfected (FIG. 4C) and staining with NKp30-Ig revealed the same phenotype. Therefore, results indicate that NKp46D2-Ig and NKp30-Ig bind to cell membrane-associated heparan sulfate. An alternative interpretation is that the binding is to a cell-surface molecule associated with heparan sulfate. Yet, this possibility is excluded by the observation that soluble heparan sulfate directly inhibits the binding of NKp46D2-Ig and NKp30-Ig to tumor cells (FIG. 2). Membrane-associated heparan sulfate proteoglycans (HSPGs) can be divided into two families: glypicans, which are attached to the plasma membrane via glycosylphosphatidylinositol (GPI) anchors, and syndecans, which are transmembrane proteins (23). The involvement of GPI-anchored proteins in the binding of NKp30-Ig and NKp46D2-Ig to tumor cells was examined. Treatment of cells with D-mannosamine (white bars) inhibits GPI-anchor formation. FIG. 4D shows that such inhibition reduced binding of NKp30-Ig and NKp46D2-Ig by 2 and 4 fold, respectively. The combined results indicate the involvement of glypicans in the binding of NKp46D2-Ig and to a lesser extent, of NKp30-Ig.

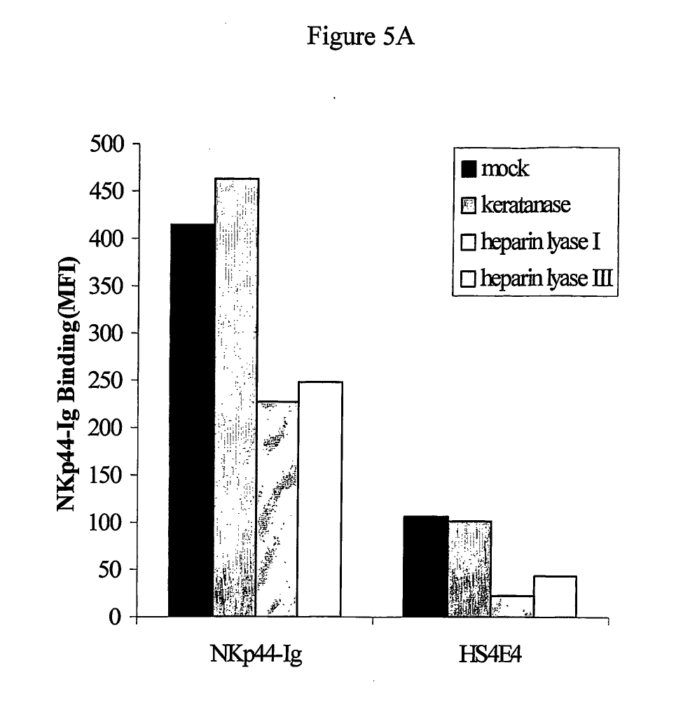

Similarly, we showed the involvement of heparan sulfate on HSPGs expressed on tumor cells for the binding of NKp44-Ig (FIG. 5). FIGS. 5B1 and 5B2 show staining of parental CHO-K1, heparan sulfate-negative and chondroitin sulfate-negative CHO cells (CHOpgsA-745) and heparan sulfate-negative and chondroitin sulfate high-positive CHO cells (CHO pgsD-677) by NKp44-Ig and human (h) second Ab (primary FACS histogram overlay), respectively. FIGS. 5B3 and 5B4 show staining of parental CHO-K1 and CHO pgsA-745 with HS4E4 (ref 35) and mouse (m) second antibody (FIGS. 5C1, 5C2, 5C3). Staining of Sham and GAS-6 cells with NKp44-Ig, HS4E4 and hIgG1, respectively (primary FACS histogram overlay). Results are from one representative experiment of two performed. For all panels, MFI results are the average of 2 different samples assayed in the same experiment. Bars, ±SD.

### Example 4

### Effect of 6-O-sulfo-N-acetylglucoseamine-PAA and Heparan Sulfate on NK Cytotoxicity

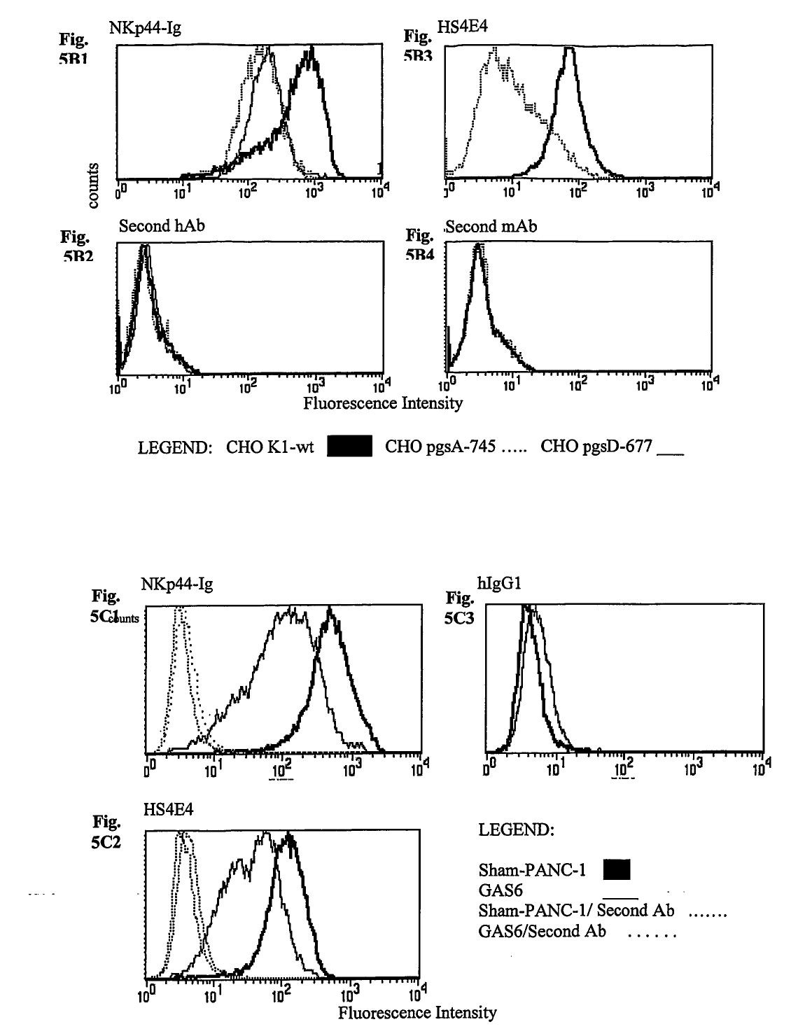

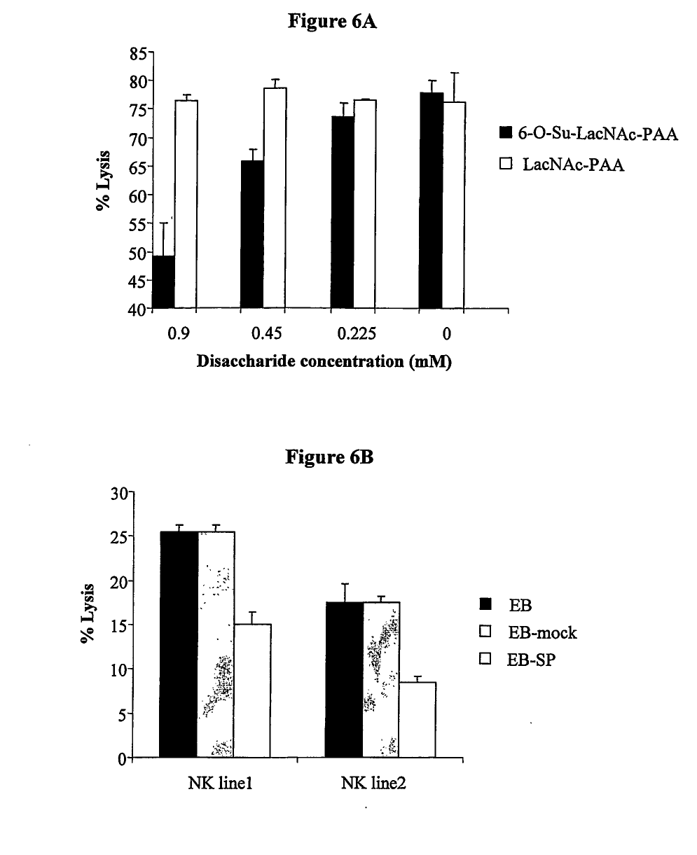

To test the role of NKp46 and NKp30 recognition of carbohydrates in NK lysis, the lysis of HeLa cells by NKs in the presence of Glyc-PAA was studied. Presence of 6-O-sulfo-LacNAc-PAA (black bars), but not LacNAc-PAA (white bars), reduced the lysis of HeLa cells by two fold (FIG. 6A). HeLa cell lysis by NK is mediated by NKp46 since specific anti-NKp46 serum, produced as described (9), reduced HeLa lysis by two fold. A concentration 0.9 mM 6-O-sulfo-LacNAc caused significant reduction of lysis (FIG. 6A). This is in the concentration range that reduced binding of NKp46D2-Ig and NKp30-Ig to HeLa and other tumor cells (FIG. 1). However, when heparin LMW was applied in order to block NK lysis, a significant two fold reduction in lysis of HeLa cells by NK was observed only at concentrations of 100 μg/ml and above (data not shown). This result is in agreement with previous publications on heparin-mediated inhibition of NK lysis (24, 25). Contrary to 6-O-sulfo-LacNAc-PAA, the heparin concentration needed for significant inhibition of lysis is at least 10 to about 1000 fold higher than the concentration used for inhibiting the binding of NKp3O-Ig and NKp46D2-Ig (FIGS. 2 and 3). A possible explanation is the plurality of heparin functions that can augment cytotoxicity while masking NKp3O and NKp46. For example, heparin efficiently potentiates the lytic activity of perforin (26). Indeed, opposite effects of heparin on NK activity, which are time and concentration dependent, were reported by Wasik and Gorski (25). Hence, a concentration-dependent balance between lysis augmenting activities of heparin and masking of NKp30 and NKp46 can result in suppression of lysis in relatively high concentrations of heparin.

Therefore, to better study the effect of target membrane-associated HSPGs on lysis by NK, the lysis of EB and EB-SP by NK cells was compared. EB-SP lysis by NK cells was reduced by 2 fold as compared to parental EB (black bars) or EB-mock cells (gray bars) (FIG. 6B). Thus, reduction in binding of NKp46-D2-Ig and NKp30-Ig to tumor cells expressing cell-surface functional heparanase is correlated with the reduced lysis of these cells by NK (FIGS. 4C and 6B).

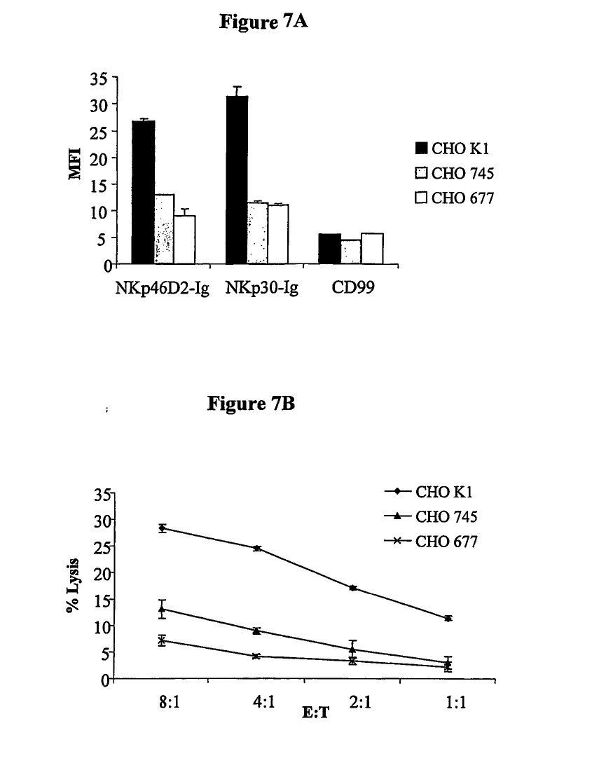

To further test the role of NKp46 and NKp30 recognition of carbohydrates in NK lysis, the lysis of CHO mutants lacking HSPG was examined. As shown in FIG. 7A, the binding of NKp46 and NKp30 to the mutant CHO cells is significantly reduced as compared to wt CHO cells. Furthermore, the lysis of CHO mutant cells lacking HSPG by NK cells is significantly lower than the lysis of the wt CHO cells (FIG. 7B). Thus, reduction in binding of NKp46-D2-Ig and NKp30-Ig to tumor cells lacking HSPG is correlated with reduced lysis of these cells by NK cells.

### Example 5

### Identification of a Region in the NKp46D2 Domain Necessary for Heparan Sulfate/Heparin Binding

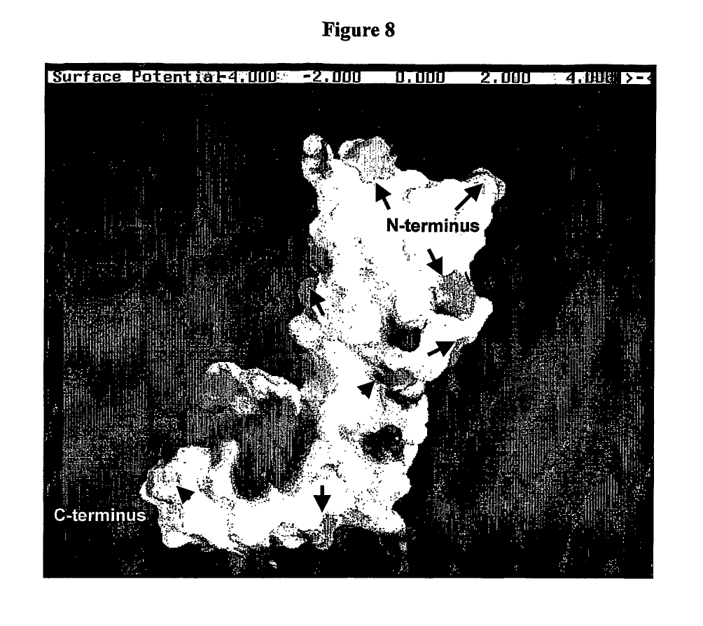

An attempt was made to identify the amino acid residues in NKp46 that are involved in heparin/heparan sulfate binding. Heparin and to a lesser extent, heparan sulfate, are negatively charged biological macromolecules due to the high content of negatively charged sulfo and carboxyl groups. Therefore, a region with a high positive surface potential could be a candidate for heparin/heparan sulfate binding. The electrostatic potential was calculated for NKp46 structure (loll PDB code) using Delphi (36) and was presented on the surface using GRASP (37). A continuous region with a positive potential was detected on the surface of D2 domain, and is mainly donated by residues Lys 157, Arg 160, His 163, Arg 166 and Lys 170 (FIG. 8). These residues reside on β strands C and C′, on the loop that connect these strands and on the loop that connects C′ strand and βstrand E (38). These results are in agreement with the fact that only D2 is essential for binding NKp46 heparieparan sulfate (39).

The assumption that this patch, having a positive potential, may be involved in heparin/heparin sulfate binding was further supported by additional data. The high folding similarity of NKp46 and the killer inhibitory receptors (KIR2LD1, KIR2LD2, KIR2LD3 with 1 im9,1 lefx and 1b6u PDB codes, respectively) has been demonstrated (38). However, running the sequence of NKp46 on the 3DPSSM threading server (40) revealed a significant sequence identity of NKp46 to that of inhibitory receptor for human natural killer cells (P58-C152 Kir) (PDB code:1nkr). This protein appears as a member of the fibronectin type III super family. Further investigating of other members in this superfamily revealed a structure of human fibronectin (FN) type III repeats 12-14 that contain two heparin binding sites (PDB code: 1 fnh). The first is a primary site (HBS-1) located in FN13, and the second is a putative secondary binding site (HBS-2) which is ˜60 Å away in FN14 (41). HBS-1 appears in the structure as a continuous positively charged patch. The involvement of its residues in heparin binding was demonstrated by biochemical and mutagenesis data (42; 43; 44) and was further supported by the fact that these residues are conserved in FNs from frog to man(45; 46; 47). The existence of a secondary heparin-binding site (HBS-2) is suggested since biochemical data indicates that both FN13 and FN14 are essential for full binding of heparin (42; 43). Specific peptides that contain part of HBS-2 residues show heparin binding ability (48; 49; 50; 51). In the crystal structure of FN12-14, HBS-2 appears as a positively charged region. This putative combining site consists of a cluster of basic residues Lys 216, Lys 219, Arg 225, Arg230 and Lys 261.

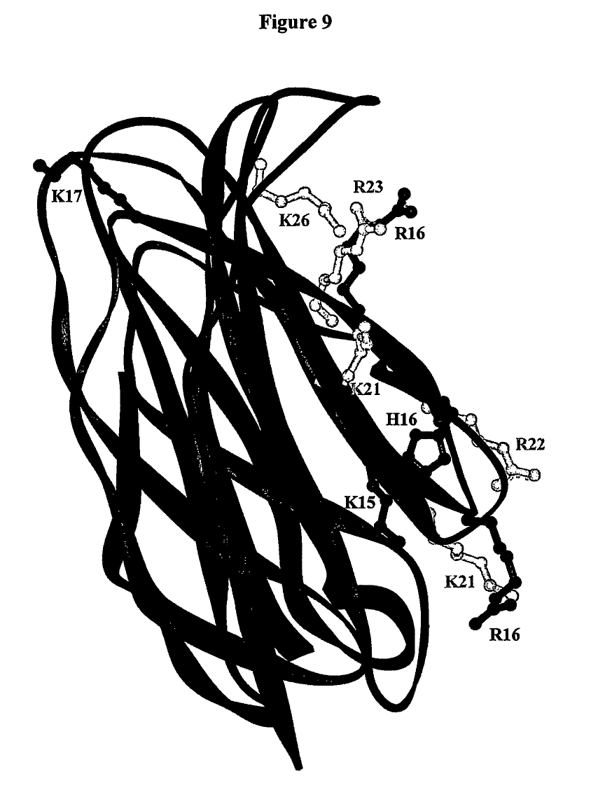

Superimposition of NKp46D2 with the FN13 structure shows no spatial overlap between HBS-1 and the residues that generate the region with the positive potential in NKp46. However, superimposition of NKp46D2 with FN14 reveals a nice spatial fit between HBS-2 and those residues that generate the region with the positive potential in NKp46 (FIG. 9). In addition to the general structural resemblance of these two regions (3.6 Årmsd for C α of residues 212-233 and 153-169 of FN14 and NKp46, respectively) the side chains of Arg 160, His 163 and Arg 166 of NKp46 reside very close to side chains of Lys 219, Arg 225 and Arg 230 of FN14, respectively. Within these regions resides the positively charged Lys 157 of the NKp46 and Lys 216 of the FN14, located at the same 3 aa distance from the Arg 160 and Lys 219, respectively. Yet, Lys 170 of NKp46, proposed to be involved in the interaction based on the electrostatic potential map (FIG. 8), does not fit with the FN14's Lys 261 (FIGS. 8, 9).

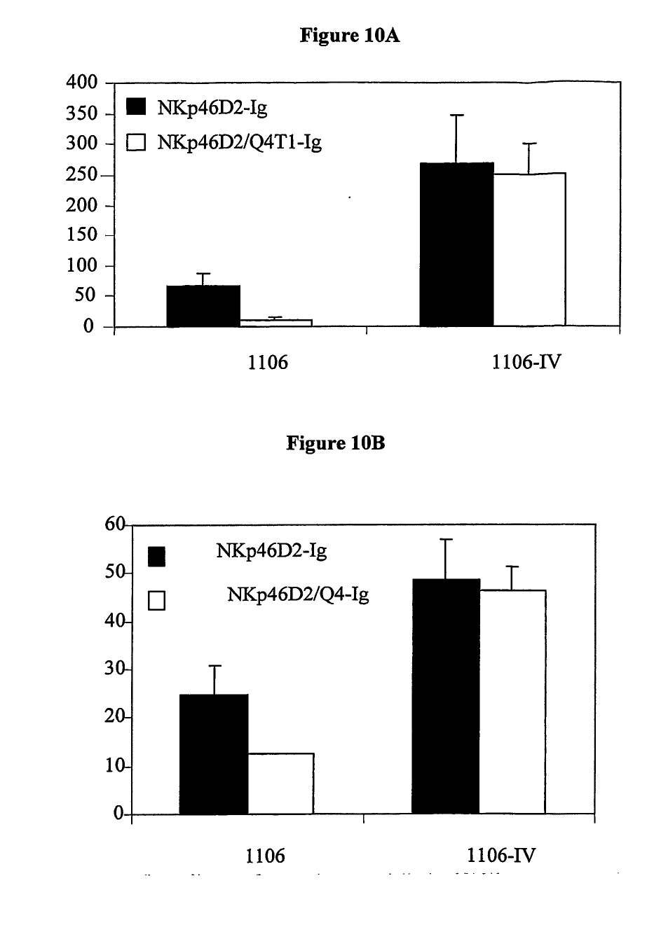

Based on the observations described above, certain site-directed mutations were made in NKp46D2 (Lys 157, Arg 160, His 163 and Arg 166; 4 point mutations) or (Lys 157, Arg 160, His 163, Arg 166, and Lys 170; 5 point mutations) into hydrophilic, neutral, amino acids with residues of similar size and to compare the ability of the mutant and the wild type receptors to bind heparin/heparan sulfate. Two constructs were prepared: Q4 (K157Q, R160Q, H163Q, and R166Q) and Q4T1 (K157Q, R160Q, H163Q, R166Q, and K170T). The mutated referred to herein as SEQ ID NO:29 SEQ ID NO:30.. Corresponding fusion proteins were prepared (NKp46D2-Q4-Ig and NKp46D2-Q4T1-Ig) and compared to NKp46D2-Ig. FIG. 10 shows that NKp46D2-Ig, NKp46D2-Q4-Ig and NKp46D2-Q4T1-Ig bind similarly to IV-infected cells. However, binding of NKp46D2-Q4-Ig and NKp46D2-Q4T1-Ig to tumor cells is significantly reduced (in particular, NKp46D2-Q4T1-Ig) as compared to NKp46D2-Ig.

The corresponding peptide sequences of NKp30 and NKp44 having SEQ ID NOS:3-5 were determined based on the electrostatic map of the NKp30and NKp44 polypeptide and sequence comparison, and synthesized accordingly.

### REFERENCES

1. Trinchieri, G. 1989. Biology of natural killer cells. Adv Immunol 47:187-376.
2. Long, E. O. 2002. Tumor cell recognition by natural killer cells. Semin Cancer Biol 12:57-61.
3. Biassoni, R., C. Cantoni, D. Pende, S. Sivori, S. Parolini, M. Vitale, C. Bottino, and A. Moretta. 2001. Human natural killer cell receptors and co-receptors. Immunol Rev 181:203-214.
4. Bakker, A. B., J. Wu, J. H. Phillips, and L. L. Lanier. 2000. NK cell activation: distinct stimulatory pathways counterbalancing inhibitory signals. Hum Immunol 61:18-27.
5. Moretta, A., C. Bottino, M. Vitale, D. Pende, C. Cantoni, M. C. Mingari, R. Biassoni, and L. Moretta. 2001. Activating receptors and coreceptors involved in human natural killer cell-mediated cytolysis. Annu Rev Immunol 19:197-223.
6. Ljunggren, H. G., and K. Karre. 1990. In search of the ′missing self: MHC molecules and NK cell recognition. Immunol Today 11:237-244.
7. McQueen, K. L., and P. Parham. 2002. Variable receptors controlling activation and inhibition of NK cells. Curr Opin Immunol 14:615-621.
8. Mandelboim, O., and A. Porgador. 2001. NKp46. Int J Biochem Cell Bio 33:1147-1150.
9. Mandelboim, O., N. Lieberman, M. Lev, L. Paul, T. I. Arnon, Y. Bushkin, D. M. Davis, J. L. Strominger, J. W. Yewdell, and A. Porgador. 2001. Recognition of haemagglutinins on virus-infected cells by NKp46 activates lysis by human NK cells. Nature 409:1055-1060.
10. Arnon, T. I., M. Lev, G. Katz, Y. Chemobrov, A. Porgador, and O. Mandelboim. 2001. Recognition of viral hemagglutinins by NKp44 but not by NKp30. Eur J Immunol 31:2680-2689.
11. Lanier, L. L. and J. H. Phillips 1996 Inhibitory MHC class I receptors on NK cells and T cells. Iinmunol. Today 17:86-91.
12. Matsuda, K. et al. 2001. Glypican-1 is overexpressed in human breast cancer and modulates the mitogenic effects of multiple heparin-binding growth factors in breast cancer cells. Cancer Res 61, 5562-9.
13. Kleeff, J. et al. 1998. The cell-surface heparan sulfate proteoglycan glypican-1 regulates growth factor action in pancreatic carcinoma cells and is overexpressed in human pancreatic cancer. J Clin Invest 102, 1662-73.
14. Sugahara, K. & Kitagawa, H. 2002. Heparin and heparan sulfate biosynthesis. IUBMB Life 54, 163-75.
15. Miyamoto, K. et al. 2003. Methylation-associated silencing of heparan sulfate D-glucosaminyl 3-O-sulfotransferase-2 (3-OST-2) in human breast, colon, lung and pancreatic cancers. Oncogene 22, 274-80.
16. Goldshmidt, O., E. Zcharia, R. Abramovitch, S. Metzger, H. Aingorn, Y. Friedmann, V. Schirmacher, E. Mitrani, and I. Vlodavsky. 2002. Cell surface expression and secretion of heparanase markedly promote tumor angiogenesis and metastasis. Proc Natl Acad Sci USA 99:10031-10036.
17. Bovin, N. V. 1998. Polyacrylamide-based glycoconjugates as tools in glycobiology. Glycoconj J 15:431-446.
18. Soker, S., D. Goldstaub, C. M. Svahn, I. Vlodavsky, B. Z. Levi, and G. Neufeld. 1994. Variations in the size and sulfation of heparin modulate the effect of heparin on the binding of VEGF165 to its receptors. Biochem Biophys Res Commun 203:1339-1347.
19. Achdout, H., T. I. Arnon, G. Markel, T. Gonen-Gross, G. Katz, N. Lieberman, R. Gazit, A. Joseph, E. Kedar, and O. Mandelboim. 2003. Enhanced recognition of human NK receptors after influenza virus infection. J Immunol 171:915-923.
20. Mandelboim, O., P. Malik, D. M. Davis, C. H. Jo, J. E. Boyson, and J. L. Strominger. 1999. Human CD16 as a lysis receptor mediating direct natural killer cell cytotoxicity. Proc Natl Acad Sci USA 96:5640-5644.
21. Desai, U. R., H. M. Wang, and R. J. Linhardt. 1993. Specificity studies on the heparin lyases from Flavobacterium heparinum. Biochemistry 32:8140-8145.
22. Sugahara, K., and H. Kitagawa. 2002. Heparin and heparan sulfate biosynthesis. IUBMB Life 54:163-175.
23. Bernfield, M., M. Gotte, P. W. Park, O. Reizes, M. L. Fitzgerald, J. Lincecum, and M. Zako. 1999. Functions of cell surface heparan sulfate proteoglycans. Annu Rev Biochem 68:729-777.
24. Yamamoto, H., S. Fuyama, S. Arai, and F. Sendo. 1985. Inhibition of mouse natural killer cytotoxicity by heparin. Cell Immunol 96:409-417.
25. Wasik, M., and A. Gorski. 1994. Heparin enhances generation of natural killer activity in vitro. Arch Immunol Ther Exp (Warsz) 42:73-76.
26. Ishiura, S., H. Koizumi, T. Tsukahara, and H. Sugita. 1988. Effects of heparin and other acid mucopolysaccharides on the activity of a cytolytic pore-forming protein (perforin) from cytotoxic T-lymphocytes. J Biochem (Tokyo) 103:11-13.
27. Kelley, L. A., MacCallum, R. M. & Stemberg, M. J. Enhanced genome annotation using structural profiles in the program 3D-PSSM. J Mol Biol 299, 499-520 (2000).
28. Sharma, A., Askari, J. A., Humphries, M. J., Jones, E. Y. & Stuart, D. I. 1999. Crystal structure of a heparin- and integrin-binding segment of human fibronectin. Embo J 18, 1468-79.
29. Capila, I., and R. J. Linhardt. 2002. Heparin-protein interactions. Angew Chem Int Ed Engl 41:391-412.
30. Porgador, A., O. Mandelboim, N. P. Restifo, and J. L. Strominger. 1997. Natural killer cell lines kill autologous beta2-microglobulin-deficient melanoma cells: implications for cancer immunotherapy. Proc Natl Acad Sci USA 94:13140.
31. Kleeff, J., S. Wildi, A. Kumbasar, H. Friess, A. D. Lander, and M. Korc. 1999. Stable transfection of a glypican-1 antisense construct decreases tumorigenicity in PANC-1 pancreatic carcinoma cells. Pancreas 19:281.
32. Esko, J. D. 1991. Genetic analysis of proteoglycan structure, function and metabolism. Curr Opin Cell Biol 3:80.
33. Mandelboim, O., N. Lieberman, M. Lev, L. Paul, T. I. Amon, Y. Bushkin, D. M. Davis, J. L. Strominger, J. W. Yewdell, and A. Porgador. 2001. Recognition of haemagglutinins on virus-infected cells by NKp46 activates lysis by human NK cells. Nature 409:1055.
34. Arnon, T. I., H. Achdout, N. Lieberman, R. Gazit, T. Gonen-Gross, G. Katz, A. Bar-Ilan, N. Bloushtain, M. Lev, A. Joseph, E. Kedar, A. Porgador, and O. Mandelboim. 2003. The mechanisms controlling the recognition of tumor and virus infected cells by NKp46. Blood 103(2):664-72.
35. Dennissen, M. A., G. J. Jenniskens, M. Pieffers, E. M. Versteeg, M. Petitou, J. H. Veerkamp, and T. H. van Kuppevelt. 2002. Large, tissue-regulated domain diversity of heparan sulfates demonstrated by phage display antibodies. J Biol Chem 277:10982.
36. Nicholls, A., and Honig, B. 1991. A Rapid Finite Difference Algorithm, Utilizing Successive Over-Relaxation to Solve the Poisson-Boltzmann Equation. J Comput Chem 12:435-445.
37. Nicholls, A., Sharp, K. A., and Honig, B. 1991. Protein folding and association: insights from the interfacial and thermodynamic properties of hydrocarbons. Proteins 11:281-296.
38. Ponassi, M., Cantoni, C., Biassoni, R., Conte, R., Spallarossa, A., Pesce, A., Moretta, A., Moretta, L., Bolognesi, M., and Bordo, D. (2003) Biochem Biophys Res Commun 309:317-323.
39. Bloushtain-N., Qimron-U, Bar-Ilan-A, Hershkovitz-O, Fima-E, Korc-M., Vlodavsky-I, Bovin-N V, Porgador-A. 2004. Membrane-Associated Heparan Sulfate Proteoglycans are Involved in the Recognition of Cellular Targets by NKp30 andNKp46. J. Immunology. 173:2392-2401.
40. Kelley, L. A., MacCallum, R. M., and Stemberg, M. J. 2000. Enhanced genome annotation using structural profiles in the program 3D-PSSM. J Mol Biol 299:499-520.
41. Sharma, A., Askari, J. A., Humphries, M. J., Jones, E. Y., and Stuart, D. I. 1999. Crystal structure of a heparin- and integrin-binding segment of human fibronectin. EMBO J 18:1468-1479.
42. Ingham, K. C., S. A. Brew, and D. H. Atha. 1990. Interaction of heparin with fibronectin and isolated fibronectin domains. Biochem. J 272:605-611.
43. Barkalow, F. J., and Schwarzbauer, J. E. 1991. Localization of the major heparin-binding site in fibronectin. J Biol Chem 266:7812-7818.
44. Busby, T. F., Argraves, W. S., Brew, S. A., Pechik, I., Gilliland, G. L., and Ingham, K. C. 1995. Heparin binding by fibronectin module III-13 involves six discontinuous basic residues brought together to form a cationic cradle. J Biol Chem 270:18558-18562.
45. Hynes, R. O., Schwarzbauer, J. E., and Tamkun, J. W. 1984. Fibronectin: a versatile gene for a versatile protein. Ciba Found Symp 108:75-92.
46. DeSimone, D. W., Spiegel, E., and Spiegel, M. 1985. The biochemical identification of fibronectin in the sea urchin embryo. Biochem Biophys Res Commun 133:183-188.
47. Norton, P. A., and Hynes, R. O. 1987. Alternative splicing of chicken fibronectin in embryos and in normal and transformed cells Mol Cell Biol 7:4297-4307.
48. McCarthy, J. B., Chelberg, M. K., Mickelson, D. J., and Furcht, L. T. 1988. Localization and chemical synthesis of fibronectin peptides with melanoma adhesion and heparin binding activities. Biochemistry 27:1380-1388.
49. McCarthy, J. B., Skubitz, A. P., Qi, Z., Yi, X. Y., Mickelson, D. J., Klein, D. J., and Furcht, L. T. 1990. RGD-independent cell adhesion to the carboxy-terminal heparin-binding fragment of fibronectin involves heparin-dependent and -independent activities. J Cell Biol 110:777-787.
50. Drake, S. L., Vamum, J., Mayo, K. H., Letourneau, P. C., Furcht, L. T., and McCarthy, J. B. 1993. Structural features of fibronectin synthetic peptide FN-C/H II, responsible for cell adhesion, neurite extension and heparan sulfate binding. J Biol Chem 268:15859-15867.
51. Woods, A., McCarthy, J. B., Furcht, L. T., and Couchman, J. R. 1993. A synthetic peptide from the COOH-terminal heparin-binding domain of fibronectin promotes focal adhesion formation. Mol Biol Cell 4:605-613.
52. Carpino, L A and Han, G. Y. 1972. The 9-fluorenylmethoxycarbonyl amino-protecting group. J. Org. Chem 37:3404-3409.
53. Coller, B. S., Springer, K. T., Scudder, L. E., Kutok, J. L., Ceruso, M., Prestwich G. D. 1993. Substituting isoserine for serine in the thrombin receptor activation peptide SFLLRN confers resistance to aminopeptidase M-induced cleavage and inactivation. J Biol Chem. 268(28):20741-3.
54. Powell, M. F., Stewart, T., Otvos, L. Jr, Urge, L., Gaeta, F. C., Sette, A., Arrhenius, T., Thomson, D., Soda, K., Colon, S. M. 1993. Peptide stability in drug development. II. Effect of single amino acid substitution and glycosylation on peptide reactivity in human serum. Pharm Res. 10(9):1268-73.
55. Kohler, G., Milstein, C. 1975. Continuous cultures of fused cells secreting antibody of predefined specificity. Nature 7;256(5517):495-7.
56. Anderson P, Blue M L, O'Brien C, Schlossman S F.1989. Monoclonal antibodies reactive with the T cell receptor zeta chain: production and characterization using a new method. J Immunol. 43(6):1899-904.
   It will be appreciated by a person skilled in the art that the present invention is not limited by what has been particularly shown and described hereinabove. Rather, the scope of the invention is defined by the claims that follow.

1. An isolated peptide fragment of a natural cytotoxicity receptor (NCR) of natural killer (NK) cells, active fragments, analogs or derivatives thereof, the peptide fragment capable of binding to a membrane-associated biomolecule of a tumor cell, the biomolecule comprising at least one sulfated polysaccharide, said biomolecule serving as the binding site of the NCR mediating the lysis of tumor cells by NK cells, with the proviso that said peptide is other than a full length NCR polypeptide or an isolated NCR extracellular domain.

2. The peptide fragment of claim 1 comprising about 7 to about 120 contiguous amino acids.

3. The peptide fragment of claim 1 comprising about 8 to about 100 contiguous amino acids.

4. The peptide fragment of claim 1 comprising less than about 50 contiguous amino acids.

5. The peptide of claim 1, wherein the peptide is a fragment of NCR wherein the NCR is selected from the group consisting of NKp44, NKp30 and NKp46.

6. The peptide of claim 5, wherein the peptide is a fragment of the D2 domain of NKp46 is selected from SEQ ID No:1 and SEQ ID No:2.

7. The peptide of claim 5, wherein said peptide is a fragment of NKp30 selected from SEQ ID No:3 and SEQ ID No:4.

8. The peptide of claim 5, wherein said peptide is a fragment of NKp44 having SEQ ID No: 5.

9. The peptide of claim 1, wherein said membrane-associated biomolecule is selected from a glycosaminoglycan and a proteoglycan.

10. The peptide of claim 9, wherein the glycosaminoglycan is selected from heparin, heparan sulfate and dermatan sulfate.

11. (canceled)

12. A pharmaceutical composition comprising an isolated peptide fragment according to claim 1.

13-14. (canceled)

15. The pharmaceutical composition of claim 12, the isolated peptide fragment comprising less than about 50 contiguous amino acids.

16. The pharmaceutical composition of claim 12, wherein the peptide is a fragment of NCR wherein the NCR is selected from the group consisting of NKp44, NKp30 and NKp46.

17. The pharmaceutical composition of claim 16, wherein the peptide is a fragment of the D2 domain of NKp46 selected from SEQ ID No: 1 and SEQ ID No: 2.

18. The pharmaceutical composition of claim 16, wherein said peptide is a fragment of NKp30 selected from SEQ ID No: 3 and SEQ ID No: 4.

19. The pharmaceutical composition of claim 16, wherein said peptide is a fragment of NKp44 having SEQ ID No: 5.

20-22. (canceled)

23. An antibody that recognizes an epitope on a target membrane-associated bio-molecule of a tumor cell, the biomolecule comprising at least one sulfated polysaccharide, said biomolecule mediating the lysis of tumor cells by NK cells via the natural cytotoxicity receptor (NCR).

24. The antibody of claim 23, wherein the membrane-associated biomolecule is selected from a glycosaminoglycan and a proteoglycan.

25-26. (canceled)

27. A pharmaceutical composition comprising an antibody according to claim 23.

28-30. (canceled)

31. A method of targeting a tumor cell in a subject in need thereof via an NCR-dependent mechanism, said method comprising administering to the subject a pharmaceutical composition according to any one of claims 12 or 27.

32. The method of claim 31, wherein the peptide is a fragment of NCR wherein the NCR is selected from the group consisting of NKp44, NKp30 and NKp46.

33. The method of claim 32, wherein the peptide is a fragment of the D2 domain of NKp46 is selected from SEQ ID No: 1 and SEQ ID NO:2

34. The method of claim 32, wherein the peptide is a fragment of NKp30 selected from a peptide having SEQ ID No. 3 and SEQ ID No. 4.

35. The method of claim 32, wherein the peptide is a fragment of NKp44 having SEQ ID No. 5.

36-38. (canceled)

39. A method of identifying peptides derived from NCR which are capable of binding to a membrane-associated sulfated polysaccharide of a tumor cell, comprising the steps of:
providing a set of candidate peptides;
contacting the peptides with a tumor cell;
determining the binding of said peptides to said tumor cell; and
isolating said bound peptides.

40. (canceled)
